(19) United States
(12) Patent Application Publication
(10) Pub. No.: US 2025/0387443 A1
(43) Pub. Date: Dec. 25, 2025

(54) ENHANCING ONCOLYTIC VIROTHERAPY WITH A COMBINATION OF NUCLEAR EXPORT INHIBITOR AND MYXOMA VIRUS ACTIVATING CELL DEATH PATHWAYS

(71) Applicant: Arizona Board of Regents on behalf of Arizona State University, Scottsdale, AZ (US)

(72) Inventor: Mohammed Masmudur Rahman, Chandler, AZ (US)

(21) Appl. No.: 19/247,415

(22) Filed: Jun. 24, 2025

Related U.S. Application Data
(60) Provisional application No. 63/663,515, filed on Jun. 24, 2024.

(51) Int. Cl. A61K 35/768; A61K 9/00; A61K 31/497; A61P 35/00; C07K 14/54; C07K 14/705; C07K 14/715; C12N 7/00

(52) U.S. Cl. CPC A61K 35/768; A61K 9/0019; A61K 9/0053; A61K 31/497; A61P 35/00; C07K 14/5443; C07K 14/70578; C07K 14/7155; C12N 7/00; C12N 2710/24022; C12N 2710/24032

(57) ABSTRACT

Disclosed herein are compositions and methods for treating cancer. The methods can comprise administrating to a subject with cancer a therapeutically effective amount of an oncolytic virus or modified oncolytic virus and a therapeutically-effective amount of a nuclear export inhibitor. Methods disclosed herein can convert nonpermissive or semi-permissive cancers to permissive cancers that are susceptible to infection and killing by oncolytic viruses or modified oncolytic viruses.

### CROSS-REFERENCE TO RELATED APPLICATIONS

This application claims the benefit of U.S. Provisional Application No. 63/663,515, filed Jun. 24, 2024, the disclosure of which is incorporated herein by reference in its entirety.

### REFERENCE TO A “SEQUENCE LISTING,” SUBMITTED AS AN XML FILE

The Sequence Listing written in the xml file titled: “206339-0120-00US_SequenceListing.xml”; created on Jun. 24, 2025, and 18,604 bytes in size, is hereby incorporated by reference.

### STATEMENT AS TO FEDERALLY SPONSORED RESEARCH

This invention was made with government support under R01 AI080607 awarded by the National Institutes of Health. The government has certain rights in the invention.

### BACKGROUND

Oncolytic viruses, such as from the Poxviridae family of viruses, can be mammalian viruses that are designed and/or selected for their ability to selectively infect and kill transformed cancer cells, and by their ability to activate host's immune system against the virus and also tumor antigens. However, the application of oncolytic viruses can be limited in certain tumor cells, for example nonpermissive tumor cells. Therefore, there remains a need to improve therapies based on oncolytic viruses.

### SUMMARY

Disclosed herein, in some aspects, is a method of treating cancer in a subject in need thereof, the method comprising administering to the subject an effective amount of a myxoma virus (MYXV) and an effective amount of a nuclear export inhibitor.

Disclosed herein, in some aspects, is a method of treating cancer in a subject in need thereof, the method comprising administering to the subject an effective amount of a MYXV and an effective amount of a nuclear export inhibitor, wherein the MYXV is genetically modified to express a heterologous transgene, lacks expression for a gene, or a combination thereof.

In some embodiments, the genetically modified MYXV expresses interleukin-15 and interleukin-15 receptor alpha heterodimer complex (IL15/IL15Rα), human tumor necrosis factor superfamily member 14 (LIGHT), lacks expression for M11L, or a combination thereof. In some embodiments, the genetically modified MYXV expresses IL15/IL15Rα (v-Myx-IL15Rα). In some embodiments, the genetically modified MYXV expresses human LIGHT (v-Myx-hLIGHT). In some embodiments, the genetically modified MYXV lacks expression for M11L (v-Myx-M11LKO). In some embodiments, the genetically modified MYXV expresses IL15/IL15Rα and lacks expression for M11L (vMyx-M11LKO-IL15Rα).

In some embodiments, the nuclear export inhibitor is administered orally.

Disclosed herein, in some aspects, is a method of treating cancer in a subject in need thereof, the method comprising administering to the subject an effective amount of a MYXV and an effective amount of a nuclear export inhibitor, wherein the nuclear export inhibitor is selinexor and is administered at a dose per kilogram of subject body weight of between about 0.001 mg/kg and about 1000 mg/kg.

In some embodiments, the nuclear export inhibitor: is a selective inhibitor of nuclear export (SINE), binds to and/or inhibits exportin 1 (XPO1/CRM1), binds to and/or inhibits a factor that binds to a nuclear export signal, binds to and/or inhibits a factor that binds to RAN, RAN-GTP, and/or RAN-GDP, binds to and/or inhibits a factor that docks to the nuclear pore complex, and/or binds to and/or inhibits a factor that mediates leucine-rich nuclear export signal (NES)-dependent protein transport.

In some embodiments, the nuclear export inhibitor is not rapamycin or a structural analog thereof. In some embodiments, the nuclear export inhibitor is at least one selected from the group consisting of selinexor, leptomycin A, leptomycin B, ratjadone A, ratjadone B, ratjadone C, ratjadone D, anguinomycin A, goniothalamin, piperlongumine, plumbagin, curcumin, valtrate, acetoxychavicol acetate, prenylcoumarin osthol, KOS 2464, PKF050-638, or CBS9106.

In some embodiments, the nuclear export inhibitor is selinexor and is administered at a dose per kilogram of subject body weight of between about 0.001 mg/kg and about 1000 mg/kg. In some embodiments, selinexor is administered in a tablet or a capsule. In some embodiments, at least two doses of selinexor are administered.

In some embodiments, the MYXV is administered locally, systemically, intratumorally, intravenously, via injection, or via infusion. In some embodiments, the MYXV is administered at a dose of from about 1×10³ focus-forming units (FFU) to about 1×10¹⁴ FFU. In some embodiments, at least two doses of the MYXV are administered. In some embodiments, the MYXV and selinexor are administered simultaneously or sequentially.

In some embodiments, the method increases replication of the MYXV in cancer cells of the subject by at least 10%. In some embodiments, the method is effective to reduce average cancer load by at least 10% and/or prolong survival by at least 5% relative to an otherwise comparable treatment regimen that lacks either the MYXV or selinexor as determined by a cohort study. In some embodiments, the cancer load comprises a tumor volume or circulating hematological cancer cells. In some embodiments, upon local administration of the MYXV, the MYXV reduces incidence of metastasis at a site distal from the site of administration at least 10% more than in a corresponding method that lacks either the MYXV or selinexor as determined by a cohort study.

In some embodiments, the heterologous transgene encodes a cytokine, interleukin, cell matrix protein, antibody, checkpoint inhibitor, multi-specific immune cell engager, or a functional fragment thereof. In some embodiments, the heterologous transgene encodes an anti-PD-L1 antibody, decorin, IL-12, LIGHT, p14 FAST, TNF-α, a functional fragment thereof, or a combination thereof. In some embodiments, the multi-specific immune cell engager is a bispecific killer cell engager (BiKE) or a bispecific T cell engager (BiTE).

In some embodiments, the cancer is selected from the group consisting of a solid tumor, hematological tumor, sarcoma or a carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, adenocarcinoma, pancreatic cancer, and melanoma.

In some embodiments, the subject is immunocompetent, immunocompromised, or immunodeficient.

In some embodiments, the subject is a mammal. In some embodiments, the subject is a human. In some embodiments, the method further comprises adsorbing the MYXV to a leukocyte ex vivo and administering the leukocyte to the subject.

Disclosed herein, in some aspects, is a therapeutic regimen comprising administering a myxoma virus (MYXV) and a nuclear export inhibitor to a subject with cancer, wherein the therapeutic regimen is effective to reduce average cancer load by at least 5% and/or prolong average survival by at least 5% relative to an otherwise comparable treatment regimen that lacks either the MYXV or the nuclear export inhibitor as determined by a cohort study. In some embodiments, the nuclear export inhibitor is administered orally.

In some embodiments, the MYXV is genetically modified. In some embodiments, the MYXV is genetically modified to express a heterologous transgene, lacks expression for a gene, or a combination thereof. In some embodiments, the genetically modified MYXV expresses interleukin-15 and interleukin-15 receptor alpha heterodimer complex (IL15/IL15Rα), human LIGHT, lacks expression for M11L, or a combination thereof. In some embodiments, the genetically modified MYXV expresses IL15/IL15Rα (v-Myx-IL15Rα). In some embodiments, the genetically modified MYXV expresses human LIGHT (v-Myx-hLIGHT). In some embodiments, the genetically modified MYXV lacks expression for M11L (v-Myx-M11LKO). In some embodiments, the genetically modified MYXV expresses IL15/IL15Rα and lacks expression for M11L (vMyx-M11LKO-IL15Rα).

In some embodiments, the nuclear export inhibitor: is a selective inhibitor of nuclear export (SINE), binds to and/or inhibits exportin 1 (XPO1/CRM1), binds to and/or inhibits a factor that binds to a nuclear export signal, binds to and/or inhibits a factor that binds to RAN, RAN-GTP, and/or RAN-GDP, binds to and/or inhibits a factor that docks to the nuclear pore complex, and/or binds to and/or inhibits a factor that mediates leucine-rich nuclear export signal (NES)-dependent protein transport. In some embodiments, the nuclear export inhibitor is not rapamycin or a structural analog thereof. In some embodiments, the nuclear export inhibitor is at least one selected from the group consisting of selinexor, leptomycin A, leptomycin B, ratjadone A, ratjadone B, ratjadone C, ratjadone D, anguinomycin A, goniothalamin, piperlongumine, plumbagin, curcumin, valtrate, acetoxychavicol acetate, prenylcoumarin osthol, KOS 2464, PKF050-638, or CBS9106.

In some embodiments, the nuclear export inhibitor is selinexor and is administered at a dose per kilogram of subject body weight of between about 0.0001 mg/kg and about 1000 mg/kg. In some embodiments, selinexor is administered at a dose of between about 0.01 mg/kg and about 100 mg/kg.

In some embodiments, the MYXV is administered locally, systemically, intratumorally, intravenously, via injection, or via infusion. In some embodiments, the MYXV is administered at a dose of from about 1×10³ focus-forming units (FFU) to about 1×10¹⁴ FFU. In some embodiments, the MYXV and selinexor are administered simultaneously or sequentially. In some embodiments, the cancer load comprises a tumor volume or concentration of circulating hematological cancer cells. In some embodiments, the therapeutic regimen is effective to reduce the average cancer load by at least about 20% and/or prolong average survival by at least about 20% relative to an otherwise comparable treatment regimen. In some embodiments, the MYXV is administered locally and the therapeutic regimen reduces incidence of metastasis at least 10% more than in a corresponding treatment regimen that lacks selinexor as determined by a cohort study and/or reduces cancer growth at a site distal from the site of administration at least 10% more than in a corresponding treatment regimen that lacks selinexor as determined by a cohort study.

In some embodiments, the cancer is selected from the group consisting of a solid tumor, hematological tumor, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, colorectal adenocarcinoma, pancreatic cancer, and melanoma.

In some embodiments, the subject is immunocompetent, immunocompromised, or immunodeficient. In some embodiments, the subject is a mammal. In some embodiments, the subject is a human. In some embodiments, the therapeutic regimen further comprises adsorbing the MYXV to a leukocyte ex vivo and administering the leukocyte to the subject.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a schematic illustrating oncolytic MYXV replication in human cancer cells is regulated by diverse cellular factors and signaling pathways.

FIG. 2A through FIG. 2F depict representative experimental results demonstrating that selinexor treatment significantly enhanced MYXV gene expression and replication in diverse human cancer cell lines. FIG. 2A, FIG. 2B, and FIG. 2C depict representative immunofluorescent images of PANC-1, Colo205, and MDA-MB435 cells, respectively, treated with different concentration of selinexor for 1 hour, infected with different MOI of vMyx-GFP-TdTomato for 1 hour, and replaced with fresh media containing the same doses of selinexor. Fluorescence images were taken 48 hours post-infection. FIG. 2D, FIG. 2E, and FIG. 2F depict representative results from experiments where PANC-1, Colo205, and MDA-MB435 cells, respectively, were harvested at different times post infection to determine progeny virus production by titration assays on permissive RK13 cells. Data represent mean±SD and n=3. Statistically significant differences in comparison to infection without selinexor was observed.

FIG. 3A through FIG. 3C depict representative experimental results demonstrating that combination of selinexor and oncolytic MYXV significantly reduced human cancer cell proliferation. FIG. 3A depicts a schematic representation of the cell proliferation assay protocol using Click-iT™ EdU Cell Proliferation Kit for Imaging, Alexa Fluor™ 594 dye. FIG. 3B depicts representative immunofluorescent images of PANC-1 cells left untreated (mock) or treated with selinexor, vMyx-GFP, or a combination of selinexor+vMyx-GFP. Cell proliferation assay was performed using Click-iT EdU kit. The images were taken using a fluorescence microscope. The assays were performed in triplicate and a representative is shown. FIG. 3C depicts a representative quantification of the number of cells showing labelling with EdU 594. A minimum of 100 cells were used for analysis from fluorescence images taken in FIG. 3B. Data represent mean±SD and n=3. Statistically significant differences among the different treatments are indicated. **P<0.01, ***P<0.001.

FIG. 4A and FIG. 4B depict representative experimental results demonstrating that selinexor treatment significantly enhanced replication of vMyx-M11L-KO and vMyx-hLIGHT viruses in nonpermissive human PANC-1 cancer cell line. PANC-1 cells were treated with different concentration of selinexor for 1 h, infected with an MOI of 1 with vMyx-M11L-KO or vMyx-hLIGHT viruses for 1 h and replaced with fresh media containing the same doses of selinexor. Cells were harvested at different times post infection to determine progeny virus production by titration assays on permissive RK13 cells. FIG. 4A depicts representative experimental results with vMyx-M11L-KO. FIG. 4B depicts representative experimental results with vMyx-hLIGHT. Data represent mean±SD and n=3.

FIG. 5A through FIG. 5D depict representative experimental results demonstrating that combination of selinexor and oncolytic MYXV significantly reduced viability of human cancer cells. PANC-1 cells were seeded in 96 well plates. Next day cells were left untreated (mock); treated with 1 μM or 0.1 μM of selinexor; infected with an MOI of 5 or an MOI of 1 of vMyx-M11LKO; or treated with a combination of selinexor and vMyx-M11LKO for the indicated time points. Cell viability was measured using the MTS agent assay reagents. FIG. 5A depicts a quantification from experiments with 1 μM selinexor+/−vMyx-M11LKO MOI 5. FIG. 5B depicts a quantification from experiments with 1 μM selinexor+/−vMyx-M11LKO MOI 1. FIG. 5C depicts a quantification from experiments with 0.1 μM selinexor+/−vMyx-M11LKO MOI 5. FIG. 5D depicts a quantification from experiments with 0.1 μM selinexor+/−vMyx-M11LKO MOI 1. Data represent mean±SD and n=4. The assays were performed in triplicate and a representative is shown. Statistically significant differences among the different treatments are indicated. ns P>0.05, *P<0.05, **P<0.01, ***P<0.001.

FIG. 6A and FIG. 6B depict representative experimental results demonstrating that combination of selinexor and oncolytic MYXVs significantly reduced PANC-1 xenograft tumor burden and prolong survival in NSG mice. FIG. 6A depicts a diagram of experimental setup. NSG mice were inoculated with 1×10⁶ PANC-1 cells at day 0 via SQ injection on one of the flanks. After 2-3 weeks, animals received four doses of selinexor by oral gavage and four Intratumoral (IT) injection of either vMyx-M11LKO or vMyx-hLIGHT. FIG. 6B depicts a quantification of tumor volumes on the flank were measured at different days after the start of treatments. Results are presented as mean±SEM.

FIG. 7A through FIG. 7C depict representative experimental results demonstrating that selinexor in combination with oncolytic MYXV significantly reduced HT29 xenograft tumor burden in NSG mice. FIG. 7A depicts a diagram of experimental setup. NSG mice were inoculated with 1×10⁶ HT29 cells at day 0 via SQ injection on both flanks. After 2-3 weeks animals were separated into different treatment groups. Animals received either four doses of selinexor by oral gavage (G2) or four doses of IT injection of different viruses on the right flank or a combination of selinexor by oral gavage and IT injection of viruses on the right flank. FIG. 7B and FIG. 7C depict quantification of tumor volumes on the left and the right flanks, respectively, measured on different days after the start of treatments. Results are presented as mean±SEM.

FIG. 8A through FIG. 8C depict representative experimental results demonstrating that combination of selinexor and oncolytic MYXVs significantly reduced CT26 (Mouse colon adenocarcinoma) xenograft tumor burden and prolong survival in immunocompetent BALB/c mice. FIG. 8A depicts a diagram of experimental setup. BALB/c mice were inoculated with 1×10⁶ CT26 cells at day 0 via SQ injection on the right hind flank. After tumor volume reached ˜100 mm³, animals received three doses of selinexor by oral gavage and four IT injection of either vMyx-M11LKO or vMyx-IL15Rα. FIG. 8B depicts a quantification of tumor volumes measured at every 2-3 days after the start of treatments. Results are presented as mean±SEM. FIG. 8C depicts a graph illustrating that combination therapy significantly enhances survival of mice.

FIG. 9A through FIG. 9C depict representative experimental results demonstrating that selinexor in combination with oncolytic MYXV significantly reduced CT26 xenograft tumor burden in immunocompetent BALB/c mice. FIG. 9A depicts a diagram of experimental setup. BALB/c mice were inoculated with 1×10⁶ CT26 cells at day 0 via SQ injection on the right hind flank. After the tumor volume reached ˜100 mm³ the mice received either three doses of selinexor by oral gavage and/or four intratumoral doses of vMyx-M11LKO-IL15Rα. FIG. 9B show tumor volumes measured every 2-3 days after the start of treatments. Results are presented as mean±SEM. FIG. 9C demonstrates enhanced survival of mice treated with either selinexor alone or the combination with vMyx-M11LKO-IL15Rα.

### DETAILED DESCRIPTION

The ability of viruses to infect and replicate in host cells can vary based on cell species, cell type, and other cell attributes. Oncolytic viruses selectively or preferentially replicate in cancer cells, however while some cancer cells can be permissive to a given oncolytic virus, others may be only semi-permissive, or non-permissive, reducing the efficacy of the oncolytic virus as a therapeutic. The present disclosure provides compositions and methods for converting non-permissive or semi-permissive cancer cells into permissive cells, promoting replication of the oncolytic virus and modified oncolytic viruses in the cancer cells and thereby enhancing cancer cell killing and/or anti-cancer immunity.

As demonstrated herein, nuclear export pathways can restrict viral replication, and inhibition of nuclear export pathways can enhance viral replication in such semi-permissive and non-permissive human cancer cells. Additionally, modified oncolytic viruses can be used in combination with inhibitors of nuclear export pathways to enhance cancer killing.

### Compositions

In some embodiments, the present invention provides compositions comprising an oncolytic virus. In some embodiments, the oncolytic virus is a myxoma virus. In some embodiments, the myxoma virus is a modified myxoma virus.

In some embodiments, the modified myxoma virus has been modified to knock-out at least one native gene. In some embodiments, the modified myxoma virus has been modified so that at least one native gene is non-functional. In some embodiments, the modified myxoma virus has been modified to remove at least one native genes. In some embodiments, the modified myxoma virus has been modified to knock out at least anti-apoptotic gene.

In some embodiments, the modified myxoma virus has been modified to express at least one transgene. In some embodiments, the at least one transgene encodes at least one protein that reduces cancer cell proliferation.

In some embodiments, the modified myxoma virus has been modified to knock-out at least one native gene and to express at least one transgene. In some embodiments, the modified myxoma virus has been modified to knock-out at least one anti-apoptotic gene and to express at least one transgene that encodes a protein that reduces cancer cell proliferation.

In some embodiments, the composition further comprises at least one nuclear export inhibitor. In some embodiments, the composition comprises a modified myxoma virus and at least one nuclear export inhibitor. In some embodiments, the composition comprises a modified myxoma virus that has at least one anti-apoptotic gene knocked out and expresses at least one transgene that encodes a protein that reduces cancer cell proliferation and at least one nuclear export inhibitor.

### Oncolytic Viruses

Compositions and methods of the disclosure utilize oncolytic viruses. In some embodiments, an oncolytic virus is a mammalian virus that is engineered and/or selected for its ability to selectively infect and kill cancer cells, and for an ability to activate the host immune system against the virus and/or tumor antigens.

An oncolytic virus described herein can be a virus capable of selectively or preferentially replicating in cancer cells. An oncolytic virus described herein can be a virus capable of selectively or preferentially replicating in dividing cells (e.g., a proliferative cell such as a cancer cell). Infection of and replication in a cancer cell can slow the growth of the proliferative cell and/or kill the proliferative cell, while showing no, substantially no, or less replication in non-dividing cells. An oncolytic virus can contain a viral genome packaged into a viral particle or virion and can be infectious (e.g., capable of entering into a host cell or subject). An oncolytic virus can be a DNA virus. An oncolytic virus can be an RNA virus.

An oncolytic virus can be a poxvirus from the Poxviridae family. Poxviruses are double-stranded DNA viruses that collectively are capable of infecting both vertebrates and invertebrates. Members of the Poxviridae family of viruses are a diverse group of large, complex double-stranded DNA viruses that can replicate in the cytoplasm of infected permissive cells. The genomes of most poxviruses are about 150,000 to 300,000 base pairs in length and encode approximately 150 to 300 proteins. About half of these viral proteins can be highly conserved between different poxvirus members and perform essential functions like cell binding and entry, genome replication, transcription and virion assembly. Other viral proteins can be involved in evading many host defense functions, for example, can be required for the inhibition or manipulation of diverse intracellular anti-viral signaling pathways functioning in the cytoplasm and nucleus. The poxviral genes can be expressed in distinct phases. For example, the early gene products can include proteins that are necessary for viral DNA replication and are expressed before the DNA is replicated. Intermediate/late gene products expressed during or after DNA replication can include the structural proteins required for virion maturation. Some evidence suggests that the steps of this complex viral replication process (starting from un-coating the genome, early gene expression, DNA replication, late gene expression and an even more complex virion maturation processes) can occur exclusively in the cytoplasm of the infected cells. However, there is also evidence that host cell proteins from cytoplasm and nuclear compartments participate in at least some steps of poxvirus replication. Many diverse cellular proteins and signaling pathways have been implicated in defending the cell against the infection and replication of poxviruses.

Poxviruses include, for example, species and genera of viruses that are classified as being a part of the Chordopoxvirinae subfamily such as Orthopoxvirus, Parapoxvirus, Avipoxvirus, Capripoxvirus, Leporipoxvirus, Suipoxvirus, Molluscipoxvirus, and Yatapoxvirus genera, and the Entomopoxvirinae subfamily, including Alphaentomopoxvirus, Betaentomopoxvirus, and Gammaentopoxvirus genera.

In some embodiments, the poxvirus is genetically modified. In some embodiments, the poxvirus is a Leporipoxvirus. In some embodiments, the Leporipoxvirus is a myxoma virus (MYXV). In some embodiments, the poxvirus is an Orthopoxvirus. In some embodiments, the Orthopoxvirus is a vaccinia virus. In some embodiments, the vaccinia virus is a vaccinia virus strain selected from the group consisting of Lister, Wyeth, Western Reserve, Modified Vaccinia virus Ankara, and LC16m series. In some embodiments, the Orthopoxvirus is a Raccoonpox virus. In some embodiments, the poxvirus is a Capripox virus. In some embodiments, the Capripox virus is an Orf virus.

In some embodiments, the oncolytic virus is a myxoma virus (MYXV) or is derived from a MYXV. MYXV is a member of the family poxviridae and genus Leporipoxvirus. In some embodiments, the MYXV is a wild-type strain of MYXV or is derived from a wild-type strain of MYXV. In some embodiments, the MYXV is a genetically modified strain of MYXV or is derived from a genetically modified strain of MYXV. In some instances, the MYXV is Lausanne strain or is derived from Lausanne strain. In some instances, the MYXV is a South American MYXV strain that circulates in Sylvilagus brasiliensis or is derived from a South American MYXV strain that circulates in Sylvilagus brasiliensis. In some instances, the MYXV is a Californian MYXV strain that circulates in Sylvilagus bachmani or is derived from a Californian MYXV strain that circulates in Sylvilagus bachmani. In some instances, the MYXV is 6918, an attenuated Spanish field strain that comprises modifications in genes M009L, M036L, M135R, and M148R (GenBank Accession number EU552530 which is hereby incorporated by reference as provided by GenBank on Jul. 27, 2019) or is derived from 6918. In some instances, the MYXV is 6918VP60-T2 (GenBank Accession Number EU552531 which is hereby incorporated by reference as provided by GenBank on Jul. 27, 2019) or is derived from 6918VP60-T2. In some instances, the MYXV is a strain termed the Standard laboratory Strain (SLS) or is derived from SLS.

In some embodiments, the MYXV is able to preferentially, or selectively, infect and kill permissive human cancer cells derived from different tissues. In normal primary human cells, the replication of MYXV can be restricted by multiple factors such as, for example, the cellular binding determinants, the intracellular anti-viral signaling pathways, type I IFN signaling pathways, and/other cytokine-mediated cellular anti-viral states. In human cancer cells, these self-defense cell pathways are commonly defective. MYXV replication in some human cancer cells can depend on cellular RNA helicase family proteins. Without wishing to be bound by any particular theory, RNA helicases which shuttle between nuclear and cytoplasmic compartments of cells may influence MYXV replication in virus-infected cells. Beside RNA helicases, other nuclear proteins may contribute to the replication cycle of MYXV and other poxviruses. For example, nuclear proteins might affect the replication efficiency of poxviruses in transformed human host cell lines.

MYXV late gene expression, replication, and progeny virus formation can be limited in certain human cancer cells or cancer cell types. These cancer cells and cancer cell types can be classified as semi-permissive and non-permissive human cancer cells.

In some embodiments, the oncolytic virus is from a virus family consisting of: Poxviridae, Herpesviridae, Reoviridae, Paramyxoviridae, Retroviridae, Adenoviridae, Rhabdoviridae, Picornaviridae, Parvoviridae, and Picornaviridae, or is derived from a virus family consisting of: Poxviridae, Herpesviridae, Reoviridae, Paramyxoviridae, Retroviridae, Adenoviridae, Rhabdoviridae, Picornaviridae, Parvoviridae, and Picornaviridae. In some embodiments, the oncolytic virus is from the Herpesviridae family or is derived from the Herpesviridae family. In some embodiments, the oncolytic virus is from the Reoviridae family or is derived from the Reoviridae family. In some embodiments, the oncolytic virus is from the Paramyxoviridae family or is derived from Paramyxoviridae family. In some embodiments, the oncolytic virus is from the Retroviridae family or is derived from is from the Retroviridae family. In some embodiments, the oncolytic virus is from the Adenoviridae family or is derived from the Adenoviridae family. In some embodiments, the oncolytic virus is from the Rhabdoviridae family or is derived from the Rhabdoviridae family. In some embodiments, the oncolytic virus is from the Picornaviridae family or is derived from the Picornaviridae family. In some embodiments, the oncolytic virus is from the Parvoviridae family or is derived from the Parvoviridae family. In some embodiments, the oncolytic virus is from the Picornaviridae family. In some embodiments, the oncolytic virus is from a genus that is Simplexvirus, Rubulavirus, or Senecavirus or is derived from a genus that is Simplexvirus, Rubulavirus, or Senecavirus. In some embodiments, the oncolytic virus is from genus Simplexvirus or is derived from genus Simplexvirus. In some embodiments, the oncolytic virus is from genus Rubulavirus or is derived from genus Rubulavirus. In some embodiments, the oncolytic virus is from genus Senecavirus or is derived from genus Senecavirus. In some embodiments, the oncolytic virus is from a species of virus that is Measles, Fowlpox, Vesicular Stomatitis Virus, Mumps rubulavirus, Coxsackie Virus, and Vaccinia or is derived from a species of virus that is Measles, Fowlpox, Vesicular Stomatitis Virus, Mumps rubulavirus, Coxsackie Virus, and Vaccinia. In some embodiments, the oncolytic virus is a Measles virus or is derived from a Measles virus. In some embodiments, the oncolytic virus is a Fowlpox virus or is derived from a Fowlpox virus. In some embodiments, the oncolytic virus is a Vesicular Stomatitis Virus or is derived from a Vesicular Stomatitis Virus. In some embodiments, the oncolytic virus is a Mumps rubulavirus or is derived from Mumps rubulavirus. In some embodiments, the oncolytic virus is a Coxsackie Virus or is derived from is a Coxsackie Virus. In some embodiments, the oncolytic virus is a Vaccinia virus or is derived from is a Vaccinia virus.

In some embodiments, the oncolytic virus is a virus from Chordopoxvirinae subfamily or Entomopoxvirinae subfamily or is derived from Chordopoxvirinae subfamily or Entomopoxvirinae subfamily. In some embodiments, the oncolytic virus is from a genus that is Orthopoxvirus, Cervidpoxvirus, Parapoxvirus, Avipoxvirus, Capripoxvirus, Leporipoxvirus, Suipoxvirus, Molluscipoxvirus, Yatapoxvirus, Alphaentomopoxvirus, Betaentomopoxvirus, or Gammaentopoxvirus. In some embodiments, the oncolytic virus is derived from a virus from a genus that is Orthopoxvirus, Cervidpoxvirus, Parapoxvirus, Avipoxvirus, Capripoxvirus, Leporipoxvirus, Suipoxvirus, Molluscipoxvirus, Yatapoxvirus, Alphaentomopoxvirus, Betaentomopoxvirus, or Gammaentopoxvirus. In some embodiments, the oncolytic virus is from genus Orthopoxvirus or is derived from a virus of the genus Orthopoxvirus. In some embodiments, the oncolytic virus is a vaccinia virus or is derived from a vaccinia virus. In some embodiments, the vaccinia virus is a vaccinia virus strain selected from the group consisting of Lister, Wyeth, Western Reserve, Modified Vaccinia virus Ankara, and LC16m series. In some embodiments, the oncolytic virus is a Raccoonpox virus or is derived from a Raccoonpox virus. In some embodiments, the oncolytic virus is from genus Cervidpoxvirus or is derived from a virus of the genus Cervidpoxvirus. In some embodiments, the oncolytic virus is an Orf virus or is derived from an Orf virus. In some embodiments, the oncolytic virus is from genus Parapoxvirus or is derived from a virus of the genus Parapoxvirus. In some embodiments, the oncolytic virus is from genus Avipoxvirus or is derived from a virus of the genus Avipoxvirus. In some embodiments, the oncolytic virus is from genus Capripoxvirus or is derived from a virus of the genus Capripoxvirus. In some embodiments, the oncolytic virus is from genus Suipoxvirus or is derived from a virus of the genus Suipoxvirus. In some embodiments, the oncolytic virus is from genus Molluscipoxvirus or is derived from a virus of the genus Molluscipoxvirus. In some embodiments, the oncolytic virus is from genus Yatapoxvirus or is derived from a virus of the genus Yatapoxvirus. In some embodiments, the oncolytic virus is from genus Alphaentomopoxvirus or is derived from a virus of the genus Alphaentomopoxvirus. In some embodiments, the oncolytic virus is from genus Betaentomopoxvirus or is derived from a virus of the genus Betaentomopoxvirus. In some embodiments, the oncolytic virus is from genus Gammaentopoxvirus or is derived from a virus of the genus Gammaentopoxvirus. In some embodiments, the oncolytic virus is from genus Leporipoxvirus or is derived from a virus of the genus Leporipoxvirus. In some embodiments, the oncolytic virus is replication-competent.

### Genetic Modifications

The oncolytic virus genome can include at least one modification. Modifications to the oncolytic virus genome include, but are not limited to, gene modifications, gene deletions, gene disruptions, and insertion of therapeutic transgenes. Modifications to the genome may be made using molecular biology techniques known to a skilled person, and described for example in Sambrook et al. ((2001) Molecular Cloning: a Laboratory Manual, 3rd ed., Cold Spring Harbour Laboratory Press). A skilled person will be able to determine which portions of the oncolytic viral genome can be deleted such that the virus is still capable of productive infection, for example, to provide a replication competent virus. For example, non-essential regions of the viral genome that can be deleted can be deduced from comparing the published viral genome sequence with the genomes of other well-characterized viruses (see for example C. Cameron, S. Hota-Mitchell, L, Chen, J. Barrett, J.-X. Cao, C. Macaulay, D. Willer, D. Evans, and G. McFadden, Virology (1999) 264: 298-318)).

In some embodiments, the disclosed modified oncolytic virus is a candidate to treat human malignancies. In some embodiments, the oncolytic genome comprises at least one gene modification, deletion and/or disruption. In some embodiments, the oncolytic virus comprises at least one transgene.

In some embodiments, an oncolytic virus of the disclosure comprises at least one gene modification, deletions, and/or disruptions in the viral genome. For example, an oncolytic virus of the disclosure can comprise at least one insertions, deletions, or substitutions within or adjacent to at least one gene in the genome. An insertion, deletion or modification can comprise a gene knockout (for example, deletion of at least one nucleotide that prevent functionality of the product encoded by the gene, or insertion of at least one nucleotides that disrupt expression and/or function of the product encoded by the gene). In some embodiments, an insertion, deletion, or modification does not comprise a gene knockout (for example, a sequence can be inserted at an intergenic locus between two genes, without disrupting expression of the two genes).

In some embodiments, an oncolytic virus of the disclosure comprises at least one insertions, deletions, or substitutions within or adjacent to at least one gene associated with the ability of the virus to cause discase in a host animal. In some embodiments, an oncolytic virus of the disclosure comprises at least one insertions, deletions, or substitutions within or adjacent to at least one gene associated with host cell tropism. In some embodiments, an oncolytic virus of the disclosure comprises at least one insertions, deletions, or substitutions within or adjacent to at least one gene associated with the ability of the virus to evade an innate immune response. In some embodiments, an oncolytic virus of the disclosure comprises at least one insertions, deletions, or substitutions within or adjacent to at least one gene that modulate immune signaling in an infected cell (e.g., cytokine receptor signaling). In some embodiments, an oncolytic virus of the disclosure comprises at least one insertions, deletions, or substitutions within or adjacent to at least one gene that modulate a cell death pathway in an infected cell (e.g., a gene that codes for a product that promotes or inhibits apoptosis, such as myxoma virus gene M11L). In some embodiments, an oncolytic virus of the disclosure comprises at least one insertions, deletions, or substitutions within or adjacent to at least one gene that modulates viral replication in a cancer cell (e.g., increases or decreases the rate of viral replication in a cancer cell).

In some embodiments, the modified oncolytic virus is a modified myxoma virus (MYXV).

In some embodiments, the MYXV comprises a modification to at least one gene associated with the ability of the virus to cause disease in a host animal, associated with host cell tropism, associated with the ability of the virus to evade an innate immune response, that can modulate immune signaling in an infected cell, that can modulate a cell death pathway in an infected cell, that can modulate viral replication in a cancer cell, or a combination thereof, comprise any at least one of M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD. In some embodiments, the modified MYXV comprises a modification to the MILL gene. In some embodiments, the modified MYXV is a M11L-knockout MYXV (vMyx-M11LKO).

In some embodiments, a MYXV of the disclosure comprises a modification of a MYXV gene. In some instances, the modification is a deletion that impairs the function of a protein encoded by the MYXV gene. In some cases, the modification is a partial deletion (e.g., a 10%, 20%, 30%, 40%, 50%, 60%, 70%, 80%, 90%, or 95% deletion) of the MYXV gene. In other cases, the modification is a full deletion of the MYXV gene. In some embodiments, the modification is a replacement of the MYXV gene with at least one transgene.

In some embodiments, a modified oncolytic virus comprises at least one transgene. A heterologous transgene can be selected to enhance the anticancer effect of an oncolytic virus. In some embodiments a heterologous transgene triggers cell death, for example, apoptosis, necrosis, or necroptosis. In some embodiments a heterologous transgene targets the infected cell for immune destruction, such as a gene that repairs a lack of response to interferon, or that results in the expression of a cell surface marker that stimulates an antibody response, such as a bacterial cell surface antigen. In some embodiments a heterologous transgene reduces a cancer cell's proliferation.

In some embodiments, the heterologous transgene encodes a cytokine or a functional fragment thereof, for example, IL-12, IL-15, IL-15Rα, IL15/IL15Rα (a fusion protein of interleukin-15 (IL15) and IL15 receptor alpha), tumor necrosis factor superfamily member 14 (TNFSF14; hereinafter “LIGHT”), p14 FAST, TNF-α. In some embodiments, the heterologous transgene encodes an interleukin or a functional fragment thereof, for example, IL-12, IL-15, or IL15/IL15Rα. In some embodiments, the heterologous transgene encodes IL15/IL15Rα or a functional fragment thereof. In some embodiments, the heterologous transgene encodes IL15/IL15Rα. In some embodiments, the heterologous transgene encodes a cell matrix protein or a functional fragment thereof, for example, decorin. In some embodiments, the heterologous transgene encodes an antibody or a functional fragment thereof, for example, an anti-PD-L1 or anti-PD-1 antibody or antigen-binding fragment thereof, or another immune checkpoint inhibitor. In some embodiments, the heterologous transgene encodes an anti-PD-L1 antibody, decorin, IL-12, LIGHT, p14 FAST, TNF-α, a functional fragment thereof, or a combination thereof. In some embodiments, the heterologous transgene encodes LIGHT. In some embodiments, LIGHT is human LIGHT. In some embodiments, the heterologous transgene encodes human LIGHT. In some embodiments, the heterologous transgene encodes a checkpoint inhibitor or a functional fragment thereof. In some embodiments, the heterologous transgene encodes a multi-specific immune cell engager, for example, a bispecific killer cell engager (BiKE) or a bispecific T cell engager (BiTE). In some embodiments, the heterologous transgene encodes IL15/IL15Rα and LIGHT.

In some embodiments, the oncolytic virus is modified to comprise a heterologous transgene and to delete or disrupt at least one endogenous viral genes. In some embodiments, the modified oncolytic virus is a MYXV. In some embodiments, the MYXV is modified to comprise a heterologous transgene encoding IL15/IL15Rα (vMyx-IL15Rα). In some embodiments, the MYXV is modified to comprise a heterologous transgene encoding a fragment comprising IL15 from IL15/IL15Rα. In some embodiments, the MYXV is modified to comprise a heterologous transgene encoding a fragment comprising IL15 from IL15/IL15Rα, comprising an amino acid sequence with at least 90%, at least 91%, at least 92%, at least 93%, at least 94%, at least 95%, at least 96%, at least 97%, at least 98%, or at least 99% homology to SEQ ID NO:5 or SEQ ID NO:11. In some embodiments, the MYXV is modified to comprise a heterologous transgene encoding a fragment comprising IL15 from IL15/IL15Rα, comprising the amino acid sequence of SEQ ID NO:5 or SEQ ID NO:11. In some embodiments, the MYXV is modified to comprise a heterologous transgene encoding a fragment comprising IL15 from IL15/IL15Rα, comprising a nucleotide sequence with at least 90%, at least 91%, at least 92%, at least 93%, at least 94%, at least 95%, at least 96%, at least 97%, at least 98%, or at least 99% homology to SEQ ID NO:6 or SEQ ID NO:12. In some embodiments, the MYXV is modified to comprise a heterologous transgene encoding a fragment comprising IL15 from IL15/IL15Rα, comprising the nucleotide sequence of SEQ ID NO:6 or SEQ ID NO:12.

In some embodiments, the MYXV is modified to comprise a heterologous transgene encoding a fragment comprising IL15Rα from IL15/IL15Rα. In some embodiments, the MYXV is modified to comprise a heterologous transgene encoding a fragment comprising IL15Rα from IL15/IL15Rα, comprising an amino acid sequence with at least 90%, at least 91%, at least 92%, at least 93%, at least 94%, at least 95%, at least 96%, at least 97%, at least 98%, or at least 99% homology to SEQ ID NO:7 or SEQ ID NO:13. In some embodiments, the MYXV is modified to comprise a heterologous transgene encoding a fragment comprising IL15Rα from IL15/IL15Rα, comprising the amino acid sequence of SEQ ID NO:7 or SEQ ID NO:13. In some embodiments, the MYXV is modified to comprise a heterologous transgene encoding a fragment comprising IL15Rα from IL15/IL15Rα, comprising a nucleotide sequence with at least 90%, at least 91%, at least 92%, at least 93%, at least 94%, at least 95%, at least 96%, at least 97%, at least 98%, or at least 99% homology to SEQ ID NO:8 or SEQ ID NO:14. In some embodiments, the MYXV is modified to comprise a heterologous transgene encoding a fragment comprising IL15Rα from IL15/IL15Rα, comprising the nucleotide sequence of SEQ ID NO:8 or SEQ ID NO:14.

In some embodiments, the MYXV is modified to comprise a heterologous transgene encoding IL15/IL15Rα, comprising an amino acid sequence with at least 90%, at least 91%, at least 92%, at least 93%, at least 94%, at least 95%, at least 96%, at least 97%, at least 98%, or at least 99% homology to SEQ ID NO:1 or SEQ ID NO:9. In some embodiments, the MYXV is modified to comprise a heterologous transgene encoding IL15/IL15Rα, comprising the amino acid sequence of SEQ ID NO:1 or SEQ ID NO:9. In some embodiments, the MYXV is modified to comprise a heterologous transgene encoding IL15/IL15Rα, comprising a nucleotide sequence with at least 90%, at least 91%, at least 92%, at least 93%, at least 94%, at least 95%, at least 96%, at least 97%, at least 98%, or at least 99% homology to SEQ ID NO:2 or SEQ ID NO:10. In some embodiments, the MYXV is modified to comprise a heterologous transgene encoding IL15/IL15Rα, comprising the nucleotide sequence of SEQ ID NO:2 or SEQ ID NO:10. In some embodiments, the MYXV is modified to comprise a heterologous transgene encoding IL15/IL15Rα and to delete, disrupt, or modify M11L (vMyx-M11LKO-IL15Rα). In some embodiments, the MYXV is modified to comprise a heterologous transgene encoding IL15/IL15Rα, comprising an amino acid sequence with at least 90%, at least 91%, at least 92%, at least 93%, at least 94%, at least 95%, at least 96%, at least 97%, at least 98%, or at least 99% homology to SEQ ID NO:1 or SEQ ID NO:9, and to delete, disrupt, or modify M11L. In some embodiments, the MYXV is modified to comprise a heterologous transgene encoding IL15/IL15Rα, comprising the amino acid sequence of SEQ ID NO:1 or SEQ ID NO:9, and to delete, disrupt, or modify M11L. In some embodiments, the MYXV is modified to comprise a heterologous transgene encoding IL15/IL15Rα, comprising a nucleotide sequence with at least 90%, at least 91%, at least 92%, at least 93%, at least 94%, at least 95%, at least 96%, at least 97%, at least 98%, or at least 99% homology to SEQ ID NO:2 or SEQ ID NO:10, and to delete, disrupt, or modify M11L. In some embodiments, the MYXV is modified to comprise a heterologous transgene encoding IL15/IL15Rα, comprising the nucleotide sequence of SEQ ID NO:2 or SEQ ID NO:10, and to delete, disrupt, or modify M11L.

In some embodiments, the MYXV is modified to comprise a heterologous transgene encoding LIGHT (vMyx-LIGHT). In some embodiments, the MYXV is modified to comprise a heterologous transgene encoding LIGHT, comprising an amino acid sequence with at least 90%, at least 91%, at least 92%, at least 93%, at least 94%, at least 95%, at least 96%, at least 97%, at least 98%, or at least 99% homology to SEQ ID NO:3. In some embodiments, the MYXV is modified to comprise a heterologous transgene encoding LIGHT, comprising the amino acid sequence of SEQ ID NO:3. In some embodiments, the MYXV is modified to comprise a heterologous transgene encoding LIGHT, comprising a nucleotide sequence with at least 90%, at least 91%, at least 92%, at least 93%, at least 94%, at least 95%, at least 96%, at least 97%, at least 98%, or at least 99% homology to SEQ ID NO:4. In some embodiments, the MYXV is modified to comprise a heterologous transgene encoding LIGHT, comprising the nucleotide sequence of SEQ ID NO:4. In some embodiments, the MYXV is modified to comprise a heterologous transgene encoding LIGHT and to delete, disrupt, or modify M11L (vMyx-M11LKO-LIGHT).

In some embodiments, the MYXV is modified to comprise a heterologous transgene encoding LIGHT, comprising an amino acid sequence with at least 90%, at least 91%, at least 92%, at least 93%, at least 94%, at least 95%, at least 96%, at least 97%, at least 98%, or at least 99% homology to SEQ ID NO:3, and to delete, disrupt, or modify M11L. In some embodiments, the MYXV is modified to comprise a heterologous transgene encoding LIGHT, comprising the amino acid sequence of SEQ ID NO:3, and to delete, disrupt, or modify M11L. In some embodiments, the MYXV is modified to comprise a heterologous transgene encoding LIGHT, comprising a nucleotide sequence with at least 90%, at least 91%, at least 92%, at least 93%, at least 94%, at least 95%, at least 96%, at least 97%, at least 98%, or at least 99% homology to SEQ ID NO:4, and to delete, disrupt, or modify M11L. In some embodiments, the MYXV is modified to comprise a heterologous transgene encoding LIGHT, comprising the nucleotide sequence of SEQ ID NO:4, and to delete, disrupt, or modify M11L.

-mouse IL15/IL15Rα amino acid sequence
SEQ ID NO: 1
MKILKPYMRNTSISCYLCFLLNSHFLTEAGIHVFILGCVSVGLPKTEANWIDVRY
DLEKIESLIQSIHIDTTLYTDSDFHPSCKVTAMNCFLLELQVILHEYSNMTLNETVRNVLYLANS
TLSSNKNVAESGCKECEELEEKTFTEFLQSFIRIVQMFINTSMETDTLLLWVLLLWVPGST
GDGTTCPPPVSIEHADIRVKNYSVNSRERYVCNSGFKRKAGTSTLIECVINKNTNVA
HWTTPSLKCIRDPS
-mouse IL15/IL15Rα nucleotide sequence
SEQ ID NO: 2
ATGAAAATTTTGAAACCATATATGAGGAATACATCCATCTCGTGCTACTTGTG
TTTCCTTCTAAACAGTCACTTTTTAACTGAGGCTGGCATTCATGTCTTCATTTTGGGCTGTG
TCAGTGTAGGTCTCCCTAAAACAGAGGCCAACTGGATAGATGTAAGATATGACCTGGAGAA
AATTGAAAGCCTTATTCAATCTATTCATATTGACACCACTTTATACACTGACAGTGACTTTCA
TCCCAGTTGCAAAGTTACTGCAATGAACTGCTTTCTCCTGGAATTGCAGGTTATTTTACATG
AGTACAGTAACATGACTCTTAATGAAACAGTAAGAAACGTGCTCTACCTTGCAAACAGCAC
TCTGTCTTCTAACAAGAATGTAGCAGAATCTGGCTGCAAGGAATGTGAGGAGCTGGAGGA
GAAAACCTTCACAGAGTTTTTGCAAAGCTTTATACGCATTGTCCAAATGTTCATCAACACGT
CCATGGAGACAGACACCCTGCTGCTCTGGGTGCTGCTGCTGTGGGTGCCCGGC
TCTACCGGCGACGGCACCACCTGCCCTCCCCCTGTGTCCATCGAGCACGCCGA
CATCAGAGTGAAGAACTACTCCGTGAACTCTCGGGAGAGATACGTGTGCAACT
CCGGCTTCAAGCGGAAGGCCGGCACCTCCACCCTGATCGAGTGCGTGATCAAC
AAGAACACCAACGTGGCCCACTGGACCACCCCTTCCCTGAAGTGCATCCGGGA
CCCTTCCTGA
-human LIGHT amino acid sequence
SEQ ID NO: 3
MEESVVRPSVFVVDGQTDIPFTRLGRSHRRQSCSVARVGLGLLLLLMGAG
LAVQGWELLQLHWRLGEMVTRLPDGPAGSWEQLIQERRSHEVNPAAHLTGANSSLTGS
GGPLLWETQLGLAFLRGLSYHDGALVVTKAGYYYIYSKVQLGGVGCPLGLASTITHGL
YKRTPRYPEELELLVSQQSPCGRATSSSRVWWDSSFLGGVVHLEAGEEVVVRVLDERL
VRLRDGTRSYFGAFMV
-human LIGHT nucleotide sequence
SEQ ID NO: 4
ATGGAGGAGAGTGTCGTACGGCCCTCAGTGTTTGTGGTGGATGGACAG
ACCGACATCCCATTCACGAGGCTGGGACGAAGCCACCGGAGACAGTCGTGCAGTGT
GGCCCGGGTGGGTCTGGGTCTCTTGCTGTTGCTGATGGGGGCCGGGCTGGCCGTCCA
AGGCTGGTTCCTCCTGCAGCTGCACTGGCGTCTAGGAGAGATGGTCACCCGCCTGCC
TGACGGACCTGCAGGCTCCTGGGAGCAGCTGATACAAGAGCGAAGGTCTCACGAGG
TCAACCCAGCAGCGCATCTCACAGGGGCCAACTCCAGCTTGACCGGCAGCGGGGGG
CCGCTGTTATGGGAGACTCAGCTGGGCCTGGCCTTCCTGAGGGGCCTCAGCTACCAC
GATGGGGCCCTTGTGGTCACCAAAGCTGGCTACTACTACATCTACTCCAAGGTGCAG
CTGGGCGGTGTGGGCTGCCCGCTGGGCCTGGCCAGCACCATCACCCACGGCCTCTAC
AAGCGCACACCCCGCTACCCCGAGGAGCTGGAGCTGTTGGTCAGCCAGCAGTCACC
CTGCGGACGGGCCACCAGCAGCTCCCGGGTCTGGTGGGACAGCAGCTTCCTGGGTG
GTGTGGTACACCTGGAGGCTGGGGAGGAGGTGGTCGTCCGTGTGCTGGATGAACGC
CTGGTTCGACTGCGTGATGGTACCCGGTCTTACTTCGGGGCTTTCATGGTGTGA
-mouse IL15 from IL15/IL15Rα amino acid sequence
SEQ ID NO: 5
MKILKPYMRNTSISCYLCFLLNSHFLTEAGIHVFILGCVSVGLPKTEANWI
DVRYDLEKIESLIQSIHIDTTLYTDSDFHPSCKVTAMNCFLLELQVILHEYSNMTLNETVR
NVLYLANSTLSSNKNVAESGCKECEELEEKTFTEFLQSFIRIVQMFINTS
-mouse IL15 from IL15/IL15Rα nucleotide sequence
SEQ ID NO:6
ATGAAAATTTTGAAACCATATATGAGGAATACATCCATCTCGTGCTAC
TTGTGTTTCCTTCTAAACAGTCACTTTTTAACTGAGGCTGGCATTCATGTCTTCATTTT
GGGCTGTGTCAGTGTAGGTCTCCCTAAAACAGAGGCCAACTGGATAGATGTAAGAT
ATGACCTGGAGAAAATTGAAAGCCTTATTCAATCTATTCATATTGACACCACTTTAT
ACACTGACAGTGACTTTCATCCCAGTTGCAAAGTTACTGCAATGAACTGCTTTCTCCT
GGAATTGCAGGTTATTTTACATGAGTACAGTAACATGACTCTTAATGAAACAGTAAG
AAACGTGCTCTACCTTGCAAACAGCACTCTGTCTTCTAACAAGAATGTAGCAGAATC
TGGCTGCAAGGAATGTGAGGAGCTGGAGGAGAAAACCTTCACAGAGTTTTTGCAAA
GCTTTATACGCATTGTCCAAATGTTCATCAACACGTCC
-mouse IL15Rα from IL15/IL15Rα amino acid sequence
SEQ ID NO: 7
METDTLLLWVLLLWVPGSTGDGTTCPPPVSIEHADIRVKNYSVNSRERYV
CNSGFKRKAGTSTLIECVINKNTNVAHWTTPSLKCIRDPS
-mouse IL15Rα from IL15/IL15Rα nucleotide sequence
SEQ ID NO: 8
ATGGAGACAGACACCCTGCTGCTCTGGGTGCTGCTGCTGTGGGTGCCC
GGCTCTACCGGCGACGGCACCACCTGCCCTCCCCCTGTGTCCATCGAGCACGCCGAC
ATCAGAGTGAAGAACTACTCCGTGAACTCTCGGGAGAGATACGTGTGCAACTCCGG
CTTCAAGCGGAAGGCCGGCACCTCCACCCTGATCGAGTGCGTGATCAACAAGAACA
CCAACGTGGCCCACTGGACCACCCCTTCCCTGAAGTGCATCCGGGACCCTTCCTGA
-human IL15/IL15Rα amino acid sequence
SEQ ID NO: 9
MRISKPHLRSISIQCYLCLLLNSHFLTEAGIHVFILGCFSAGLPKTEANWVNVIS
DLKKIEDLIQSMHIDATLYTESDVHPSCKVTAMKCFLLELQVISLESGDASIHDTVENLIILANN
SLSSNGNVTESGCKECEELEEKNIKEFLQSFVHIVQMFINTSMAPRRARGCRTLGLPALLL
LLLLRPPATRGITCPPPMSVEHADIWVKSYSLYSRERYICNSGFKRKAGTSSLTECV
LNKATNVAHWTTPS
-human IL15/IL15Rα nucleotide sequence
SEQ ID NO: 10
ATGAGAATTTCGAAACCACATTTGAGAAGTATTTCCATCCAGTGCTACTTGT
GTTTACTTCTAAACAGTCATTTTCTAACTGAAGCTGGCATTCATGTCTTCATTTTGGGCTGTT
TCAGTGCAGGGCTTCCTAAAACAGAAGCCAACTGGGTGAATGTAATAAGTGATTTGAAAAA
AATTGAAGATCTTATTCAATCTATGCATATTGATGCTACTTTATATACGGAAAGTGATGTTCA
CCCCAGTTGCAAAGTAACAGCAATGAAGTGCTTTCTCTTGGAGTTACAAGTTATTTCACTTG
AGTCCGGAGATGCAAGTATTCATGATACAGTAGAAAATCTGATCATCCTAGCAAACAACAG
TTTGTCTTCTAATGGGAATGTAACAGAATCTGGATGCAAAGAATGTGAGGAACTGGAGGAA
AAAAATATTAAAGAATTTTTGCAGAGTTTTGTACATATTGTCCAAATGTTCATCAACACTTCT
ATGGCCCCGCGGCGGGCGCGCGGCTGCCGGACCCTCGGTCTCCCGGCGCTGC
TACTGCTGCTGCTGCTCCGGCCGCCGGCGACGCGGGGCATCACGTGCCCTCCC
CCCATGTCCGTGGAACACGCAGACATCTGGGTCAAGAGCTACAGCTTGTACTC
CAGGGAGCGGTACATTTGTAACTCTGGTTTCAAGCGTAAAGCCGGCACGTCCA
GCCTGACGGAGTGCGTGTTGAACAAGGCCACGAATGTCGCCCACTGGACAACC
CCCAGTCTCAAATGCA
-human IL15 from IL15/IL15Rα amino acid sequence
SEQ ID NO: 11
MRISKPHLRSISIQCYLCLLLNSHFLTEAGIHVFILGCFSAGLPKTEANWVN
VISDLKKIEDLIQSMHIDATLYTESDVHPSCKVTAMKCFLLELQVISLESGDASIHDTVEN
LIILANNSLSSNGNVTESGCKECEELEEKNIKEFLQSFVHIVQMFINTS
-human IL15 from IL15/IL15Rα nucleotide sequence
SEQ ID NO: 12
ATGAGAATTTCGAAACCACATTTGAGAAGTATTTCCATCCAGTGCTAC
TTGTGTTTACTTCTAAACAGTCATTTTCTAACTGAAGCTGGCATTCATGTCTTCATTTT
GGGCTGTTTCAGTGCAGGGCTTCCTAAAACAGAAGCCAACTGGGTGAATGTAATAA
GTGATTTGAAAAAAATTGAAGATCTTATTCAATCTATGCATATTGATGCTACTTTATA
TACGGAAAGTGATGTTCACCCCAGTTGCAAAGTAACAGCAATGAAGTGCTTTCTCTT
GGAGTTACAAGTTATTTCACTTGAGTCCGGAGATGCAAGTATTCATGATACAGTAGA
AAATCTGATCATCCTAGCAAACAACAGTTTGTCTTCTAATGGGAATGTAACAGAATC
TGGATGCAAAGAATGTGAGGAACTGGAGGAAAAAAATATTAAAGAATTTTTGCAGA
GTTTTGTACATATTGTCCAAATGTTCATCAACACTTCT
-human IL15Rα from IL15/IL15Ra amino acid sequence
SEQ ID NO: 13
MAPRRARGCRTLGLPALLLLLLLRPPATRGITCPPPMSVEHADIWVKSYSL
YSRERYICNSGFKRKAGTSSLTECVLNKATNVAHWTTPS
-human IL15Ra from IL15/IL15Rα nucleotide sequence
SEQ ID NO: 14
ATGGCCCCGCGGCGGGCGCGCGGCTGCCGGACCCTCGGTCTCCCGGCG
CTGCTACTGCTGCTGCTGCTCCGGCCGCCGGCGACGCGGGGCATCACGTGCCCTCCC
CCCATGTCCGTGGAACACGCAGACATCTGGGTCAAGAGCTACAGCTTGTACTCCAG
GGAGCGGTACATTTGTAACTCTGGTTTCAAGCGTAAAGCCGGCACGTCCAGCCTGAC
GGAGTGCGTGTTGAACAAGGCCACGAATGTCGCCCACTGGACAACCCCCAGTCTCA
AATGCAATGGCCCCGCGGCGGGCGCGCGGCTGCCGGACCCTCGGTCTCCCGGCGCT
GCTACTGCTGCTGCTGCTCCGGCCGCCGGCGACGCGGGGCATCACGTGCCCTCCCCC
CATGTCCGTGGAACACGCAGACATCTGGGTCAAGAGCTACAGCTTGTACTCCAGGG
AGCGGTACATTTGTAACTCTGGTTTCAAGCGTAAAGCCGGCACGTCCAGCCTGACGG
AGTGCGTGTTGAACAAGGCCACGAATGTCGCCCACTGGACAACCCCCAGTCTCAAA
TGCA

### Nuclear Export Inhibitors

As demonstrated herein, nuclear export inhibitors can enhance oncolytic virus and modified oncolytic virus replication and gene expression in cancer cells that are normally not susceptible, substantially not susceptible, or only exhibit limited susceptibility to infection by an oncolytic virus or modified oncolytic virus in the absence of the nuclear export inhibitor. As demonstrated herein, a treatment regimen combining an oncolytic virus or modified oncolytic virus with a nuclear export inhibitor can exhibit strikingly superior therapeutic efficacy compared to either agent alone.

It is understood and herein contemplated that there are a number of oncolytic viruses, modified oncolytic viruses, nuclear export inhibitors, dosages, and routes of administrations that can be used in the treatment of cancer. Specifically contemplated herein are genetic modifications made to oncolytic viruses that improve their replication in non-permissive cells and their ability to kill cancer cells. Oncolytic viruses, modified oncolytic viruses, nuclear export inhibitors, dosages, and routes of administrations that can be used in the treatment of cancer are described in detail in International Application No.: PCT/US23/65165 which is incorporated herein in its entirety.

A nuclear export inhibitor can be an agent that inhibits transport of molecules through the nuclear export pathway. In certain embodiments, the nuclear export inhibitor is a selective inhibitor. In certain embodiments, the nuclear export inhibitor is non-selective. In some embodiments, a nuclear export inhibitor is an agent that is capable of interfering with nucleocytoplasmic trafficking. In some embodiments, a nuclear export inhibitor alters nuclear export by interfering with protein trafficking.

Nuclear export inhibitors (NEIs) can be classified into four groups as follows: bacterial products, herbal ingredients, fungal or animal NEIs, and synthetic NEIs. Bacterial NEIs include leptomycin A/B, ratjadone A/C and anguinomycin A/B/C/D, which all have a long polyketide chain with a lactone ring. Several plant NEIs were discovered from South/Southeast Asia herbs and food additives, including valtrate, oridonin, acetoxychavicol acetate, curcumin, goniothalamin, piperlongumine and plumbagin. Wortmannin and cyclopentenone prostaglandin (15d-PGJ2) were known for other functions before they were discovered as CRM1 inhibitors. Synthetic inhibitors include PKF050-638, 5219668, SINEs, compound3/4, CBS9106 and S109.

In some embodiments, the nuclear export inhibitor comprises a selective inhibitor of the nuclear export (SINE). A SINE can be a nuclear export inhibitor that inhibits exportin 1 (XPO1/CRM1). Examples of SINEs include selinexor, KPT-185, KPT-249, KPT-251, KPT-276, KPT-330 and KPT-335.

In some embodiments, the nuclear export inhibitor inhibits exportin 1. XPO1 is a cell-cycle-regulated gene that encodes exportin 1, which mediates leucine-rich nuclear export signal (NES)-dependent protein transport. Exportin 1 mediates the nuclear export of cellular proteins (cargos) bearing a leucine-rich nuclear export signal (NES) and of RNAs. In the nucleus, in association with RANBP3, exportin 1 binds cooperatively to the NES on its target protein and to the GTPase RAN in its active GTP-bound form (Ran-GTP). Docking of this complex to the nuclear pore complex (NPC) is mediated through binding to nucleoporins. Upon transit of a nuclear export complex into the cytoplasm, disassembling of the complex and hydrolysis of Ran-GTP to Ran-GDP (induced by RANBP1 and RANGAP1, respectively) cause release of the cargo from the export receptor. The directionality of nuclear export is thought to be conferred by an asymmetric distribution of the GTP- and GDP-bound forms of Ran between the cytoplasm and nucleus. Exportin 1 is involved in U3 snoRNA transport from Cajal bodies to nucleoli. Exportin 1 binds to late precursor U3 snoRNA bearing a TMG cap. Exportin 1 can specifically inhibit the nuclear export of Rev and U snRNAs. It is involved in the control of several cellular processes by controlling the localization of cyclin B, MPAK, and MAPKAP kinase 2. Exportin 1 also regulates NFAT and AP-1.

In some embodiments, the nuclear export inhibitor binds to and/or inhibits a factor (such as a protein) that binds to a nuclear export signal. In some embodiments, the nuclear export inhibitor binds to and/or inhibits a factor that binds to RAN, RAN-GTP, and/or RAN-GDP. In some embodiments, the nuclear export inhibitor binds to and/or inhibits a factor that docks to the nuclear pore complex. In some embodiments, the nuclear export inhibitor binds to and/or inhibits a factor that mediates leucine-rich nuclear export signal (NES)-dependent protein transport.

In some embodiments, the nuclear export inhibitor comprises a covalent nuclear export inhibitor. In some embodiments, the nuclear export inhibitor comprises a non-covalent nuclear export inhibitor.

In some embodiments, the nuclear export inhibitor inhibits RNA helicase family proteins. In some embodiments, the nuclear export inhibitor inhibits an RNA helicase family protein. In some embodiments, the nuclear export inhibitor inhibits a nuclear protein. In some embodiments, the nuclear export inhibitor is an anti-cancer therapeutic.

In some embodiments, the nuclear export inhibitor comprises a small molecule compound. In some embodiments, the nuclear export inhibitor comprises a natural compound such as ratjadone, valtrate and acetoxychavicol acetate. In some embodiments, the nuclear export inhibitor comprises a reversible nuclear export inhibitor such as CBS9106.

Examples of nuclear export inhibitors include but are not limited to selinexor, leptomycin A, leptomycin B, ratjadone A, ratjadone B, ratjadone C, ratjadone D, anquinomycin A, goniothalamin, piperlongumine, plumbagin, curcumin, valtrate, acetoxychavicol acetate, prenylcoumarin osthol, KOS 2464, PKF050-638, and CBS9106. In some embodiments, a nuclear export inhibitor comprises or is trifluoperazine hydrochloride, W13, ETP-45648, Vinblastine, Akt inhibitor X, INCAs, SMIP001/004, resveratrol, ellipticine, WGA, cSN50 peptide, bimax1/2 peptide, leptomycin B, anquinomycins, goniothalamin, ratjadone, valtrate, acetoxychavicol acetate, 15d-PGJ2, peumusolide A, PKF050-638, KOS-2464, CBS9106, or a combination thereof.

In certain embodiments, the nuclear export inhibitor comprises at least one of leptomycin A, leptomycin B, ratjadone A, B, C and D, anquinomycin A, goniothalamin, piperlongumine, plumbagin, curcumin, valtrate, acetoxychavicol acetate, prenylcoumarin osthol, or synthetic nuclear export inhibitors such as KOS 2464, PKF050-638 (N-azolylacrylate analog), CBS9106, selinexor, and those found in Mathew and Ghildyal, (“CRM1 inhibitors for antiviral therapy,” Frontiers in Microbiology, Vol 8 (2017), Article 1171), which is incorporated herein by reference for such disclosure. In some embodiments, the nuclear export inhibitor comprises leptomycin A. In some embodiments, the nuclear export inhibitor comprises leptomycin B. In some embodiments, the nuclear export inhibitor comprises ratjadone A. In some embodiments, the nuclear export inhibitor comprises ratjadone B. In some embodiments, the nuclear export inhibitor comprises ratjadone C. In some embodiments, the nuclear export inhibitor comprises ratjadone D. In some embodiments, the nuclear export inhibitor comprises anquinomycin A. In some embodiments, the nuclear export inhibitor comprises goniothalamin. In some embodiments, the nuclear export inhibitor comprises piperlongumine. In some embodiments, the nuclear export inhibitor comprises plumbagin. In some embodiments, the nuclear export inhibitor comprises curcumin. In some embodiments, the nuclear export inhibitor comprises valtrate. In some embodiments, the nuclear export inhibitor comprises acetoxychavicol acetate. In some embodiments, the nuclear export inhibitor comprises prenylcoumarin osthol. In some embodiments, the nuclear export inhibitor comprises KOS 2464. In some embodiments, the nuclear export inhibitor comprises PKF050-638. In some embodiments, the nuclear export inhibitor comprises CBS9106. In some embodiments, the nuclear export inhibitor comprises or consists of selinexor. In some embodiments, the nuclear export inhibitor is selinexor

In some embodiments, the nuclear export inhibitor is not rapamycin or an analog (e.g., structural analog) thereof.

### Methods and Therapeutic Regimens

As disclosed herein, nuclear export inhibitors can be used to convert cancer cells that are not susceptible to an oncolytic virus (e.g., MYXV) or modified oncolytic virus to cancer cells that are relatively more susceptible to infection by the oncolytic virus or modified oncolytic virus, e.g., to induce susceptibility of a cancer cell to infection by the oncolytic virus or modified oncolytic virus. Addition of the nuclear export inhibitor to a method of treatment or therapeutic regimen can enhance the therapeutic efficacy of the treatment or regimen.

The nuclear export inhibitor can be used to convert a cancer cell that has low susceptibility to infection, replication, and/or killing by oncolytic virus or modified oncolytic virus into a cancer cell that has relatively higher susceptibility to infection, replication, and/or killing by oncolytic virus or modified oncolytic virus. Therefore, the nuclear export inhibitor can be administered in combination with oncolytic virus or modified oncolytic virus to improve the efficacy of the oncolytic virus or modified oncolytic virus in treating cancer and killing tumor cells.

Disclosed herein, in some embodiments, are methods of administering to a subject at least one composition of the present invention. In some embodiments, the composition comprises at least one oncolytic virus (e.g., MYXV) or modified oncolytic virus (e.g., vMyx-M11LKO, vMyx-hLIGHT, vMyx-IL15Rα, vMyx-M11LKO-IL15Rα, etc.). In some embodiments, the composition comprises an oncolytic virus or modified oncolytic virus and a therapeutically-effective amount of a nuclear export inhibitor.

In some embodiments, the method comprises administering to a subject at least one oncolytic virus (e.g., MYXV) or modified oncolytic virus (e.g., vMyx-M11LKO, vMyx-hLIGHT, vMyx-IL15Rα, vMyx-M11LKO-IL15Rα, etc.) of the present invention and a nuclear export inhibitor of the present invention. In some embodiments, the oncolytic virus or modified oncolytic virus and the nuclear export inhibitor are administered together. In some embodiments, the oncolytic virus or modified oncolytic virus and the nuclear export inhibitor are administered separately.

In some embodiments, the subject has at least one cancer. In some embodiments, the subject is a mammal. In some embodiments, the subject is a primate. In some embodiments, the subject is a human. In some embodiments, the subject is a canine. In some embodiments, the subject is a non-rodent mammal.

In one aspect, disclosed herein are methods of converting a nonpermissive or semi-permissive cell to a permissive cell comprising: contacting a cancer cell that is nonpermissive to an oncolytic virus or modified oncolytic virus with the oncolytic virus or modified oncolytic virus and a nuclear export inhibitor, thereby converting said cancer cell. In another aspect, disclosed herein are methods of killing a cancer cell comprising: contacting a cancer cell with an oncolytic virus or modified oncolytic virus and a nuclear export inhibitor, thereby killing said cancer cell. Accordingly, in some embodiments, this disclosure provides methods of increasing virus replication in a nonpermissive cancer cell by treating the nonpermissive or semi-permissive cell with a nuclear export inhibitor.

In some embodiments, the cancer cell, e.g., the nonpermissive or semi-permissive cancer cell, is an animal cell such as a mammalian cell. In some embodiments, the cancer cell, e.g., the nonpermissive cancer cell, is a human cell. In certain embodiments, the cell is an immortalized human or primate cell. In some embodiments, the cancer cell, e.g., the nonpermissive cancer cell, is a canine cell. In some embodiments, the cancer cell is a cell of a cancer tissue of a subject.

An oncolytic virus disclosed herein, such as a MYXV, or modified oncolytic virus, such as vMyx-M11LKO, vMyx-hLIGHT, vMyx-IL15Rα, vMyx-M11LKO-IL15Rα, etc., can be administered to a subject in a therapeutically-effective amount by various forms and routes including, for example, systemic, oral, topical, parenteral, intravenous injection, intravenous infusion, intratumoral injection, subcutaneous injection, intramuscular injection, intradermal injection, intraperitoneal injection, intracerebral injection, subarachnoid injection, intraspinal injection, intrasternal injection, intraarticular injection, endothelial administration, local administration, intranasal administration, intrapulmonary administration, intraarterial administration, intrathecal administration, inhalation, intralesional administration, intradermal administration, epidural administration, absorption through epithelial or mucocutaneous linings (e.g., oral mucosa, rectal and intestinal mucosa), intracapsular administration, subcapsular administration, intracardiac administration, transtracheal administration, subcuticular administration, subarachnoid administration, subcapsular administration, intraspinal administration, or intrasternal administration.

In some embodiments, the virus is administered systemically. In some embodiments, the virus is administered by injection at a disease site. In some embodiments, the virus is administered orally. In some embodiments, the virus is administered parenterally.

An oncolytic virus disclosed herein, such as a MYXV, or modified oncolytic virus, such as vMyx-M11LKO, vMyx-hLIGHT, vMyx-IL15Rα, vMyx-M11LKO-IL15Rα, etc., can be administered at any interval desired. In some embodiments, the virus can be administered hourly. In some embodiments, the virus is administered about every 1, 2, 3, 4, 5, 6, 7, 8, 9, 10, 11, 12, 13, 14, 15, 16, 17, 18, 19, 20, 22, 24, 26, 28, 30, 32, 36, 40, 44, or 48 hours. In some embodiments, the virus can be administered twice a day, once a day, five times a week, four times a week, three times a week, two times a week, once a week, once every two weeks, once every three weeks, once every four weeks, once a month, once every five weeks, once every six weeks, once every eight weeks, once every two months, once every twelve weeks, once every three months, once every four months, once every six months, once a year, or less frequently.

In some embodiments, the oncolytic virus or modified oncolytic virus, the nuclear export inhibitor, or both are administered to the subject from 1 to 4 weeks apart, for examples, about 1 week apart, about 2 weeks apart or about 3 weeks apart. In some embodiments, the oncolytic virus or modified oncolytic virus, the nuclear export inhibitor, or both are administered to the subject from 1 to 4 months apart, for examples, about 1 months apart, about 2 months apart or about 3 months apart.

An oncolytic virus disclosed herein, such as a MYXV, or modified oncolytic virus, such as vMyx-M11LKO, vMyx-hLIGHT, vMyx-IL15Rα, vMyx-M11LKO-IL15Rα, etc., can be administered in combination with at least one other therapies. An oncolytic virus (e.g., MYXV) or modified oncolytic virus (e.g., vMyx-M11LKO, vMyx-hLIGHT, vMyx-IL15Rα, vMyx-M11LKO-IL15Rα, etc.) of the disclosure can be administered in combination with a nuclear export inhibitor and in combination with at least one additional other therapies. In some embodiments, an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) of the disclosure is administered in combination with a chemotherapy, an immunotherapy, a cell therapy, a radiation therapy, a stem cell transplant (such as an autologous stem cell transplant), or a combination thereof. For example, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) can be administered either prior to or following another treatment, such as administration of radiotherapy or conventional chemotherapeutic drugs and/or a stem cell transplant, such as an autologous stem cell transplant or an allogenic stem cell transplant (e.g., a HLA-matched, HLA-mismatched, or haploidentical transplant). In some embodiments, an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) of the disclosure can be in combination with an immune checkpoint modulator.

A nuclear export inhibitor of the disclosure can be administered to a subject in an effective amount. In some embodiments, the nuclear export inhibitor is administered to a subject at a dose of about 20 mg-100 mg, about 20-60 mg, or about 60 mg-100 mg. In some embodiments, the nuclear export inhibitor is administered to a subject at a dose per kilogram of the subject's body weight, for example, at a dose of about 0.001-1000 mg/kg, about 0.01-100 mg/kg, about 5-20 mg/kg or about 0.01-10 mg/kg.

A nuclear export inhibitor of the disclosure can be administered to a subject in a therapeutically-effective amount by various forms and routes including, for example, systemic, oral, topical, parenteral, intravenous injection, intravenous infusion, intratumoral injection, subcutaneous injection, intramuscular injection, intradermal injection, intraperitoneal injection, intracerebral injection, subarachnoid injection, intraspinal injection, intrasternal injection, intraarticular injection, endothelial administration, local administration, intranasal administration, intrapulmonary administration, intraarterial administration, intrathecal administration, inhalation, intralesional administration, intradermal administration, epidural administration, absorption through epithelial or mucocutaneous linings (e.g., oral mucosa, rectal and/or intestinal mucosa), intracapsular administration, subcapsular administration, intracardiac administration, transtracheal administration, subcuticular administration, subarachnoid administration, subcapsular administration, intraspinal administration, or intrasternal administration.

In some embodiments, the nuclear export inhibitor is administered orally. In some embodiments, the nuclear export inhibitor is administered systemically. In some embodiments, the nuclear export inhibitor is administered by injection at a disease site. In some embodiments, the nuclear export inhibitor is administered parenterally.

In some embodiments, the method comprises administering a therapeutically effective amount of a nuclear export inhibitor at or near the cancer tissue. In some embodiments, the method comprises contacting the cancer cell or cancer tissue with a media comprising a therapeutically effective amount of the nuclear export inhibitor. In some embodiments, the method comprises incubating the cancer cell or cancer tissue with a composition comprising a therapeutically effective amount of the nuclear export inhibitor.

A nuclear export inhibitor of the disclosure can be administered at any interval desired. In some embodiments, the nuclear export inhibitor is administered hourly. In some embodiments, the nuclear export inhibitor is administered about every 1, 2, 3, 4, 5, 6, 7, 8, 9, 10, 11, 12, 13, 14, 15, 16, 17, 18, 19, 20, 22, 24, 26, 28, 30, 32, 36, 40, 44, or 48 hours. In some embodiments, the nuclear export inhibitor is administered twice a day, once a day, five times a week, four times a week, three times a week, two times a week, once a week, once every two weeks, once every three weeks, once every four weeks, once a month, once every five weeks, once every six weeks, once every eight weeks, once every two months, once every twelve weeks, once every three months, once every four months, once every six months, once a year, or less frequently.

A nuclear export inhibitor of the disclosure can be administered in combination with at least one other therapies. A nuclear export inhibitor of the disclosure can be administered in combination with an oncolytic virus or modified oncolytic virus and in combination with at least one additional other therapies. In some embodiments, a nuclear export inhibitor of the disclosure is administered in combination with a chemotherapy, an immunotherapy, a cell therapy, a radiation therapy, a stem cell transplant (such as an autologous stem cell transplant), or a combination thereof. For example, the nuclear export inhibitor can be administered either prior to or following another treatment, such as administration of radiotherapy or conventional chemotherapeutic drugs and/or a stem cell transplant, such as an autologous stem cell transplant or an allogenic stem cell transplant (e.g., a HLA-matched, HLA-mismatched, or haploidentical transplant). In some embodiments, a nuclear export inhibitor of the disclosure can be in combination with an immune checkpoint modulator.

In some embodiments, the method comprises a systemic administration. For example, in some embodiments, the oncolytic virus or modified oncolytic virus, the nuclear export inhibitor, or both are administered systemically.

In some embodiments, the method comprises a local administration to the cancer tissue to be treated. For example, in some embodiments, the oncolytic virus or modified oncolytic virus, the nuclear export inhibitor, or both are administered locally to a cancer tissue to be treated. In some embodiments, the oncolytic virus or modified oncolytic virus is administered locally and the nuclear export inhibitor is administered systemically. In some embodiments, the oncolytic virus or modified oncolytic virus is administered locally and the nuclear export inhibitor is administered orally. In some embodiments, the oncolytic virus or modified oncolytic virus, the nuclear export inhibitor, or both are administered parenterally. In some embodiments, the oncolytic virus or modified oncolytic virus, the nuclear export inhibitor, or both are administered by injection. In some embodiments, the oncolytic virus or modified oncolytic virus, the nuclear export inhibitor, or both are administered by cutaneous injection, subcutaneous injection, or injection to a nodal lesion. In some embodiments, the oncolytic virus or modified oncolytic virus, the nuclear export inhibitor, or both are administered by an injection to the cancer tissue or a cancer organ that contains the cancer tissue.

In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered intratumorally and the nuclear export inhibitor is administered orally. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered intravenously and the nuclear export inhibitor is administered orally. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and the nuclear export inhibitor is administered orally. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered systemically and the nuclear export inhibitor is administered orally.

In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered intratumorally and the nuclear export inhibitor is administered parenterally. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered intravenously and the nuclear export inhibitor is administered parenterally. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and the nuclear export inhibitor is administered parenterally. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered systemically and the nuclear export inhibitor is administered parenterally.

In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered intratumorally and the nuclear export inhibitor is administered locally. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered intravenously and the nuclear export inhibitor is administered locally. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and the nuclear export inhibitor is administered locally. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered systemically and the nuclear export inhibitor is administered locally.

In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered intratumorally and the nuclear export inhibitor is administered systemically. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rαa, etc.) is administered intravenously and the nuclear export inhibitor is administered systemically. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and the nuclear export inhibitor is administered systemically. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered systemically and the nuclear export inhibitor is administered systemically.

In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered intratumorally and the nuclear export inhibitor is administered topically. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered intravenously and the nuclear export inhibitor is administered topically. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and the nuclear export inhibitor is administered topically. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered systemically and the nuclear export inhibitor is administered topically.

In some embodiments, the method comprises administering the oncolytic virus or modified oncolytic virus, the nuclear export inhibitor, or both to the subject for a period of time. In some embodiments, the period of time is at least 1 day, at least 2 days, at least 3 days, at least 1 week, at least 2 weeks, at least 3 weeks, at least 4 weeks, at least 1 month, at least 3 months, at least 6 months, or at least 1 year. In some embodiments, the period of time is at most 1 day, at most 2 days, at most 3 days, at most 1 week, at most 2 weeks, at most 3 weeks, at most 4 weeks, at most 1 month, at most 3 months, at most 6 months, at most 1 year, or at most 10 years. In some embodiments, the period of time is from about 1 day to about 1 year, from about 1 month to about 12 months, or from 1 month to about 6 months.

The oncolytic virus or modified oncolytic virus and the nuclear export inhibitor can be administered together or separately. In some embodiments, the oncolytic virus or modified oncolytic virus and the nuclear export inhibitor are administered together. In some embodiments, the oncolytic virus or modified oncolytic virus and the nuclear export inhibitor are administered separately. When the oncolytic virus or modified oncolytic virus and the nuclear export inhibitor are administered separately, the oncolytic virus or modified oncolytic virus can be administered prior to the nuclear export inhibitor. When the oncolytic virus or modified oncolytic virus and the nuclear export inhibitor are administered separately, the oncolytic virus or modified oncolytic virus can be administered after the nuclear export inhibitor.

In some embodiments, the method comprises administering the oncolytic virus or modified oncolytic virus, the nuclear export inhibitor, or both according to an initial dose schedule and a subsequent dose schedule. In some embodiments, the initial dose schedule comprises a different dosing schedule from the subsequent dose schedule. In some embodiments, the initial dose schedule comprises a less frequent administration than the subsequent dose schedule. In some embodiments, the initial dose schedule comprises 1 to 10 treatments, such as 1 to 4 treatments or 2 to 3 treatments of the oncolytic virus or modified oncolytic virus, the nuclear export inhibitor, or both. In some embodiments, each treatment of the oncolytic virus or modified oncolytic virus, the nuclear export inhibitor, or both is administered from 1 week to about 6 weeks apart according to the initial dose schedule. In some embodiments, each treatment of the oncolytic virus or modified oncolytic virus, the nuclear export inhibitor, or both is administered about 1 week apart, about 2 weeks apart, about 3 weeks apart, or about 4 weeks apart according to the initial dose schedule. In some embodiments, each treatment of the oncolytic virus or modified oncolytic virus, the nuclear export inhibitor, or both is administered from 1 week to about 6 weeks apart according to the subsequent dose schedule. In some embodiments, each treatment of the oncolytic virus or modified oncolytic virus, the nuclear export inhibitor, or both is administered about 1 week apart, about 2 weeks apart, about 3 weeks apart, or about 4 weeks apart according to the subsequent dose schedule. In some embodiments, the oncolytic virus or modified oncolytic virus, the nuclear export inhibitor, or both are administered about 3 weeks apart in the initial dose schedule and about 2 weeks apart in the subsequent dose schedule.

The oncolytic virus or modified oncolytic virus and the nuclear export inhibitor can be administered to the subject with cancer simultaneously or sequentially. In some embodiments, the oncolytic virus or modified oncolytic virus and the nuclear export inhibitor are administered to the subject simultaneously. In some embodiments, the oncolytic virus or modified oncolytic virus and the nuclear export inhibitor are pre-mixed before their administration to the subject. In some embodiments, the oncolytic virus or modified oncolytic virus and the nuclear export inhibitor are administered to the subject separately. In some embodiments, the oncolytic virus or modified oncolytic virus is administered before the nuclear export inhibitor. In some embodiments, the oncolytic virus or modified oncolytic virus is administered after the nuclear export inhibitor. In some embodiments, the method comprises contacting the cancer cell or cancer tissue with the oncolytic virus or modified oncolytic virus and the nuclear export inhibitor simultaneously or sequentially. In some embodiments, the cancer cell or cancer tissue is contacted with the oncolytic virus or modified oncolytic virus before its contact with the nuclear export inhibitor. In some embodiments, the cancer cell or cancer tissue is contacted with the nuclear export inhibitor before its contact with the oncolytic virus or modified oncolytic virus. In some embodiments, the method comprises contacting the cancer cell or cancer tissue with a pre-mix of the oncolytic virus or modified oncolytic virus and the nuclear export inhibitor. In some embodiments, the cancer cell or cancer tissue is contacted with the oncolytic virus or modified oncolytic virus and the nuclear export inhibitor separately

In some embodiments, the method comprises pre-treating the cancer cell or cancer tissue with the nuclear export inhibitor. In some embodiments, the cancer cell or cancer tissue is pre-treated with the nuclear export inhibitor for at least 1 minute, at least 2 minutes, at least 5 minutes, at least 10 minutes, at least 30 minutes, at least 1 hour, at least 2 hours, at least 6 hours, at least 12 hours, at least 24 hours, or at least 1 week before contacting the cell or tissue with the oncolytic virus or modified oncolytic virus. In some embodiments, the cancer cell or cancer tissue is pre-treated with the nuclear export inhibitor for at most 1 minute, at most 2 minutes, at most 5 minutes, at most 10 minutes, at most 30 minutes, at most 1 hour, at most 2 hours, at most 6 hours, at most 12 hours, at most 24 hours, or at most 1 week before contacting the cell or tissue with the oncolytic virus or modified oncolytic virus. In some embodiments, the cancer cell or cancer tissue is pre-treated with the nuclear export inhibitor for a period of from about 1 minute to about 1 day, from about 5 minutes to about 12 hours, from about 10 minutes to about 2 hours, or from about 30 minutes to about 90 minutes before contacting the cell or tissue with the oncolytic virus or modified oncolytic virus. In some embodiments, the cancer cell or cancer tissue is pre-treated with the nuclear export inhibitor for about 1 hour before contacting the cell or tissue with the oncolytic virus or modified oncolytic virus.

The compositions can be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual subject. In certain embodiments, the therapeutic agents of the disclosure are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the therapeutic agents to be administered for the remainder of the patient's life. In some embodiments, the patient is monitored to check the progression of the disease or disorder, and the dose is adjusted accordingly. In some embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

Further disclosed is a delivery strategy where the therapeutic oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is first incubated with leukocytes ex vivo from bone marrow and/or peripheral blood mononuclear cells prior to introducing the cells into a subject with cancer. In some embodiments, the leukocytes and the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) are incubated together with a nuclear export inhibitor ex vivo. In this strategy, oncolytic virus (e.g., MYXV) or modified oncolytic virus (e.g., vMyx-M11LKO, vMyx-hLIGHT, vMyx-IL15Rα, vMyx-M11LKO-IL15Rα, etc.) may be delivered to cancer sites via migration of leukocytes pre-infected with virus ex vivo. This systemic delivery method is sometimes called “ex vivo virotherapy”, or EVV (aka EV2), because the virus is first delivered to isolated leukocytes prior to infusion into the patient. In some embodiments, the leukocytes are incubated with oncolytic virus or modified oncolytic virus (e.g., for one hour ex vivo), and then the oncolytic virus (e.g., MYXV)- or modified oncolytic virus (e.g., vMyx-M11LKO, vMyx-hLIGHT, vMyx-IL15Rα, vMyx-M11LKO-IL15Rα, etc.) loaded leukocytes are infused back into the recipient. In some embodiments, incubation with the nuclear export inhibitor increases uptake of oncolytic virus (e.g., MYXV) or modified oncolytic virus (e.g., vMyx-M11LKO, vMyx-hLIGHT, vMyx-IL15Rα, vMyx-M11LKO-IL15Rα, etc.) by the leukocytes and/or increases delivery of virus to the tumor sites. In some embodiments, the nuclear export inhibitor is not added to the leukocytes and oncolytic virus (e.g., MYXV) or modified oncolytic virus (e.g., vMyx-M11LKO, vMyx-hLIGHT, vMyx-IL15Rα, vMyx-M11LKO-IL15Rα, etc.) ex vivo. In some embodiments, the nuclear export inhibitor is administered to the subject after administering the leukocytes with adsorbed oncolytic virus or modified oncolytic virus. In some embodiments, the nuclear export inhibitor is not administered to the subject after administering the leukocytes with adsorbed oncolytic virus (e.g., MYXV) or modified oncolytic virus (e.g., vMyx-M11LKO, vMyx-hLIGHT, vMyx-IL15Rα, vMyx-M11LKO-IL15Rα, etc.).

In certain embodiments, the mononuclear leukocytes are peripheral blood cells and/or bone marrow cells obtained from the subject, for example as autologous cells. In some embodiments, the leukocytes are mononuclear peripheral blood cells and/or bone marrow cells obtained from at least one allogeneic donors, for example, a donor that is matched to the recipient for at least 1, at least 2, at least 3, at least 4, at least 5, at least 6, at least 7, or at least 8 HLA alleles (such as one or both copies of HLA-A, HLA-B, HLA-A, and/or HLA-DR alleles). HLA alleles can be typed, for example, using DNA-based methods. In some embodiments, the mononuclear peripheral blood cells and/or bone marrow cells are obtained from at least one heterologous donors.

In some embodiments, the cancer cell is allowed to incubate with the oncolytic virus (e.g., MYXV or VACV) or modified oncolytic virus (e.g., vMyx-M11LKO, vMyx-hLIGHT, vMyx-IL15Rα, vMyx-M11LKO-IL15Rα, etc.) for a period of time to allow the virus of interest to adsorb to the surface of the cell, such as about 20 minutes to about 5 hours, for example about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 2 hours, about 2.5 hours, about 3 hours, about 3.5 hours, about 4 hours, about 4.5 hours, about 5 hours, 12 hours, 18 hours, 20 hours, 24 hours, 30 hours, 36 hours, or even longer.

In some embodiments, the method comprises contacting the cancer cell or cancer tissue with the oncolytic virus or modified oncolytic virus at an MOI as described herein. The MOI can be determined for a given cell or tissue, for example, by titrating the ratio of virus to the cell or tissue, and quantifying the replication of viral progeny and/or the cell viability as disclosed herein. In some embodiments, an effective MOI can minimize drug-specific cellular toxicity, while enhancing replication of the virus and reducing cancer cell viability. In certain embodiments, the MOI of the oncolytic virus or modified oncolytic virus to the cancer cell or cancer tissue is from about 0.01 to about 10, from about 0.05 to about 5, and/or ranges therebetween.

Compositions and methods disclosed herein can be useful in methods of treating cancer in a subject in need thereof. The cancer can be a solid tumor or a blood tumor. In some embodiments, the cancer is lymphoma. In some embodiments, the cancer is leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or a solid tumor. In some embodiments, the cancer is a solid tumor. In some embodiments, the solid tumor is a sarcoma or a carcinoma. In some embodiments, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, or retinoblastoma. In some embodiments, the solid tumor is colorectal adenocarcinoma, pancreatic cancer, or melanoma. In some embodiments, the solid tumor is a bone cancer such as chondrosarcoma, Ewing sarcoma, and osteosarcoma. In some embodiments, the solid tumor is osteosarcoma. In some embodiments, the cancer is pancreatic cancer, colon cancer, or melanoma. In some embodiments, the tumor is a lymphatic tumor.

Methods of treatment and therapeutic regimens disclosed herein that combine a nuclear export inhibitor with an oncolytic virus or modified oncolytic virus can exhibit surprising and unexpected therapeutic effects.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to reduce cancer load by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, e.g., relative to before the treatment or therapeutic regimen. The reduction in cancer load can be an average reduction in cancer load as determined by a cohort study. The cancer load can comprise or can be a tumor volume, for example, a volume of one tumor or a volume of multiple tumors. The cancer load can comprise or can be a concentration of circulating hematological cancer cells.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to reduce cancer growth at a site distal from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, e.g., relative to before the treatment or therapeutic regimen. The reduction in cancer growth at a distal site can be as determined by a cohort study. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and nonetheless reduces cancer growth at the distal site.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to reduce incidence of metastasis at distal sites from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, e.g., relative to before the treatment or therapeutic regimen. The reduction in metastasis at distal sites can be as determined by a cohort study. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and nonetheless reduces metastasis at the distal sites.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to increase a rate of survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% relative to without the treatment or therapeutic regimen. The increase in survival rate can be as determined by a cohort study.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to reduce cancer load by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the nuclear export inhibitor. The reduction in cancer load can be an average reduction in cancer load as determined by a cohort study. The cancer load can comprise or can be a tumor volume, for example, a volume of one tumor or a volume of multiple tumors. The cancer load can comprise or can be a concentration of circulating hematological cancer cells.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to reduce cancer growth at a site distal from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the nuclear export inhibitor. The reduction in cancer growth at a distal site can be an average reduction in cancer growth at the distal site as determined by a cohort study. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and nonetheless reduces cancer growth at the distal site.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to reduce incidence of metastasis at distal sites from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the nuclear export inhibitor. The reduction in metastasis incidence at distal sites can be as determined by a cohort study. In some embodiments, the oncolytic virus (e.g., MYXV) or modified oncolytic virus (e.g., vMyx-M11LKO and/or vMyx-hLIGHT) is administered locally and nonetheless reduces metastasis at the distal sites.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to prolong survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold relative to an otherwise comparable treatment regimen that lacks the nuclear export inhibitor. The prolonged survival can be an increase in average survival as determined by a cohort study.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to increase a rate of survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% relative to an otherwise comparable treatment regimen that lacks the nuclear export inhibitor. The increase in survival rate can be as determined by a cohort study.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to reduce cancer load by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the oncolytic virus (e.g., lacks the MYXV) or modified oncolytic virus (e.g., lacks the vMyx-M11LKO, vMyx-hLIGHT, vMyx-IL15Rα, vMyx-M11LKO-IL15Rα, etc.). The reduction in cancer load can be an average reduction in cancer load as determined by a cohort study. The cancer load can comprise or can be a tumor volume, for example, a volume of one tumor or a volume of multiple tumors. The cancer load can comprise or can be a concentration of circulating hematological cancer cells.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to reduce cancer growth at a site distal from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the oncolytic virus (e.g., lacks the MYXV) or modified oncolytic virus (e.g., lacks the vMyx-M11LKO, vMyx-hLIGHT, vMyx-IL15Rα, vMyx-M11LKO-IL15Rα, etc.). The reduction in cancer growth at a distal site can be an average reduction in cancer growth at the distal site as determined by a cohort study. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and nonetheless reduces cancer growth at the distal site.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to reduce incidence of metastasis at distal sites from the site of administration by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold, relative to an otherwise comparable treatment regimen that lacks the oncolytic virus (e.g., lacks the MYXV) or modified oncolytic virus (e.g., lacks the vMyx-M11LKO, vMyx-hLIGHT, vMyx-IL15Rα, vMyx-M11LKO-IL15Rα, etc.). The reduction in metastasis incidence at distal sites can be as determined by a cohort study. In some embodiments, the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is administered locally and nonetheless reduces metastasis at the distal sites.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to prolong survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 3-fold, at least about 5-fold, at least about 10-fold, at least about 20-fold, at least about 30-fold, or at least about 50-fold relative to an otherwise comparable treatment regimen that lacks the oncolytic virus (e.g., lacks the MYXV) or modified oncolytic virus (e.g., lacks the vMyx-M11LKO, vMyx-hLIGHT, vMyx-IL15Rα, vMyx-M11LKO-IL15Rα, etc.). The prolonged survival can be an increase in average survival as determined by a cohort study.

In some embodiments, a method of treatment or therapeutic regimen disclosed herein that comprises a nuclear export inhibitor and an oncolytic virus (e.g., MYXV) or modified oncolytic virus (e.g., vMyx-M11LKO, vMyx-hLIGHT, vMyx-IL15Rα, vMyx-M11LKO-IL15Rα, etc.) is effective to increase a rate of survival by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% relative to an otherwise comparable treatment regimen that lacks the oncolytic virus (e.g., lacks the MYXV) or modified oncolytic virus (e.g., lacks the vMyx-M11LKO, vMyx-hLIGHT, vMyx-IL15Rα, vMyx-M11LKO-IL15Rα, etc.). The increase in survival rate can be as determined by a cohort study.

In some embodiments, the use of the nuclear export inhibitor increases the replication of the oncolytic virus (e.g., MYXV) or modified oncolytic virus (e.g., vMyx-M11LKO, vMyx-hLIGHT, vMyx-IL15Rα, vMyx-M11LKO-IL15Rα, etc.) in the cancer cell or cancer tissue. In some embodiments, the oncolytic virus or modified oncolytic virus is replicated at a rate that is at least 5%, at least 10%, at least 20%, at least 30%, at least 40%, at least 50%, at least 60%, at least 70%, at least 80%, or at least 90% faster than the replication rate in the absence of the nuclear export inhibitor. In some embodiments, the oncolytic virus or modified oncolytic virus is replicated at a rate that is at least 1.5 times, 2 times, 2.5 times, 3 times, 3.5 times, 4 times, 5 times, 6 times, 7 times, 8 times, 9 times, or 10 times faster than the replication rate in the absence of the nuclear export inhibitor. In some embodiments, the nuclear export inhibitor increases the replication of the oncolytic virus or modified oncolytic virus in the cancer cell or cancer tissue to a viral load that is at least 30%, at least 50%, at least 70%, at least 90%, at least 2-fold, at least 3-fold, at least 5-fold, at least 7-fold, at least 9-fold, at least 10-fold, at least 12-fold, or at least 15-fold higher than in the absence of the nuclear export inhibitor after 24 hours, 48 hours, 72 hours, or 96 hours post infection. In some embodiments, the nuclear export inhibitor increases the replication of the oncolytic virus or modified oncolytic virus in the cancer cell or cancer tissue by no more than 5-fold, no more than 10-fold, no more than 20-fold, no more than 50-fold or no more than 100-fold after 24 hours, 48 hours, 72 hours, or 96 hours post infection.

In some embodiments, the oncolytic virus or modified oncolytic virus is replicated at a rate that is at least 20%, at least 30%, at least 40%, at least 50%, at least 60%, at least 70%, at least 80%, or at least 90% faster than the replication rate prior to administering the nuclear export inhibitor. In some embodiments, the oncolytic virus or modified oncolytic virus is replicated at a rate that is at least 1.5 times, 2 times, 2.5 times, 3 times, 3.5 times, 4 times, 5 times, 6 times, 7 times, 8 times, 9 times, or 10 times faster than the replication rate prior to administering the nuclear export inhibitor.

In some embodiments, the use of the nuclear export inhibitor in combination with an oncolytic virus or modified oncolytic virus reduces the viability of the cancer cell or cancer tissue compared to the use of the oncolytic virus or modified oncolytic virus in the absence of the nuclear export inhibitor. In some embodiments, the cell viability is reduced by at least about 10%, at least about 20%, at least about 30%, at least about 40%, at least about 50%, at least about 60%, at least about 70%, at least about 80%, or at least about 90%. In some embodiments, the cell viability is reduced by at least about 10%, at least about 20%, at least about 30%, at least about 40%, at least about 50%, at least about 60%, at least about 70%, at least about 80%, at least about 90%, at least about 2-fold, at least about 3-fold, at least about 5-fold, at least about 7-fold, at least about 9-fold, or at least about 10-fold more compared to the cell viability when using the oncolytic virus or modified oncolytic virus in the absence of the nuclear export inhibitor.

In some embodiments, an effect (e.g., therapeutic effect) disclosed herein can be determined in a cohort study. The cohort study can utilize groups of suitable sizes to determine the effect of a treatment on a therapeutic parameter, for example, cancer load, tumor volume, concentration of circulating hematological cancer cells, cancer growth (e.g., at a distal site from the site of administration), metastasis incidence, duration of survival, rate of survival, and the like). The groups can be matched, e.g., by age, sex, and/or disease staging.

Each cohort or group can comprise a suitable number of subjects for determining the effect of a treatment on the therapeutic parameter, for example, a cohort or group can comprise at least 3, at least 4, at least 5, at least 6, at least 7, at least 8, at least 9, at least 10, at least 15, at least 20, at least 25, or at least 50 subjects.

Data can be collected at a suitable timepoint for evaluation of an effect on the therapeutic parameter at any suitable amount of time, for example, about 1 hour, about 12 hours, about 24 hours, about 48 hours, about 3 days, about 4 days, about 5 days, about 7 days, about 10 days, about 14 days, about three weeks, about four weeks, about five weeks, about six weeks, about eight weeks, about ten weeks, about 12 weeks, about 15 weeks, about 26 weeks, or about 52 weeks after a first dose or last dose of a nuclear export inhibitor or oncolytic virus or modified oncolytic virus.

### Pharmaceutical Compositions

Disclosed herein are pharmaceutical compositions comprising an oncolytic virus or modified oncolytic virus, a nuclear export inhibitor, a pharmaceutically acceptable excipient or carrier, or a combination thereof. When administered as a combination, the therapeutic agents (i.e., the oncolytic virus or modified oncolytic virus and the nuclear export inhibitor) can be formulated as separate compositions that are given at the same time or different times, or the therapeutic agents can be formulated as a single composition.

To prepare the pharmaceutical compositions according to the present disclosure, a therapeutically effective amount of at least one of the therapeutic agents can be admixed with a pharmaceutically acceptable carrier according to conventional pharmaceutical compounding techniques to produce a dose. A carrier may take a wide variety of forms depending on the form of preparation desired for administration, e.g., oral, topical ocular, or parenteral, including gels, creams ointments, lotions and time released implantable preparations, among numerous others. In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS)), and sugar alcohols such as sorbitol. Compositions suitable for parenteral administration can be enclosed in ampoules, disposable syringes or multiple dose vials made of glass or plastic. In some embodiments, formulations suitable for parenteral administration comprise aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

In some embodiments, the nuclear export inhibitor is administered as a pharmaceutically acceptable salt, complex, or prodrug. Pharmaceutically acceptable salts or complexes can refer to appropriate salts or complexes of the active compounds according to the present disclosure which retain the desired biological activity of the parent compound and exhibit limited toxicological effects to normal cells. Non-limiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

In some embodiments, the pharmaceutical composition is in a unit dosage form. In some embodiments, the unit dosage form is suitable to be administered orally. In some embodiments, the unit dosage form is suitable to be administered topically. In some embodiments, the unit dosage form is suitable to be administered intratumorally or parenterally, e.g., intravenously. Unit dosage formulations can be those containing a dose or unit, e.g., daily dose, two or three times daily dose, daily sub-dose, a weekly dose or unit, or an appropriate fraction thereof, of the administered ingredient. In some embodiments, a unit dose comprises from about 0.1 mL to about 100 mL deliverable volume, and/or ranges therebetween. In some embodiments, a unit dose comprises from about 0.5 mL to about 5 mL, from about 0.75 mL to about 2.5 mL, from about 0.9 mL to about 1.1 mL deliverable volume. In some embodiments, a unit dose comprises about 0.5 mL, about 1 mL, about 1.5 mL, or about 2 mL deliverable volume.

In some embodiments, a unit dose comprises from about 1×103 plaque-forming units (FFU) to about 1×10¹⁰ FFU of the oncolytic virus or modified oncolytic virus per mL, and/or ranges therebetween. In some embodiments, a unit dose comprises from about 1×10⁴ FFU to about 1×10⁹ FFU or from about 1×10⁶ FFU to about 1×10⁸ FFU of the oncolytic virus or modified oncolytic virus per mL, and/or ranges therebetween. In some embodiments, a unit dose comprises from about 1×10⁵ FFU to about 1×10¹⁰ FFU, from about 1×10⁶ FFU to about 1×10¹⁰ FFU, from about 1×10⁵ FFU to about 1×10¹¹ FFU, from about 1×10⁵ FFU to about 1×10⁹ FFU, from about 1×10⁶ FFU to about 1×10⁹ FFU, or from about 1×10⁷ FFU to about 1×10⁸ FFU of the oncolytic virus or modified oncolytic virus per mL, and/or ranges therebetween.

In some embodiments, a unit dose comprises about 20 mg-100 mg, about 20 mg-60 mg, or about 60 mg-100 mg of a nuclear export inhibitor. In a unit dose is per kilogram of the subject's body weight, for example, about 0.001-1000 mg/kg, about 0.01-100 mg/kg, about 5-20 mg/kg or about 0.01-10 mg/kg of a subject's body weight.

### EMBODIMENTS

Embodiment 1 is a method of treating cancer in a subject in need thereof, the method comprising administering to the subject an effective amount of a myxoma virus (MYXV) and an effective amount of a nuclear export inhibitor.

Embodiment 2 is the method of embodiment 1, wherein the MYXV is a genetically modified MYXV that expresses a heterologous transgene, does not express a native MYXV gene, or a combination thereof.

Embodiment 3 is the method of embodiment 1 or 2, wherein the genetically modified MYXV expresses interleukin-15 and interleukin-15 receptor alpha heterodimer complex (IL15/IL15Rα), expresses human tumor necrosis factor superfamily member 14 (LIGHT), lacks expression for M11L, or a combination thereof.

Embodiment 4 is the method of embodiment 2 or 3, wherein the genetically modified MYXV expresses IL15/IL15Rα (v-Myx-IL15Rα).

Embodiment 5 is the method of embodiment 4, wherein IL15/IL15Rα comprises an amino acid sequence with at least 90% homology to SEQ ID NO:1 or SEQ ID NO:9.

Embodiment 6 is the method of any one of embodiments 2-5, wherein the genetically modified MYXV expresses human LIGHT (v-Myx-hLIGHT).

Embodiment 7 is the method of any one of embodiments 2-6, wherein human LIGHT comprises an amino acid sequence with at least 90% homology to SEQ ID NO:3.

Embodiment 8 is the method of any one of embodiments 2-7, wherein the genetically modified MYXV lacks expression for M11L (v-Myx-M11LKO).

Embodiment 9 is the method of any one of embodiments 2-8, wherein the genetically modified MYXV expresses IL15/IL15Rα and lacks expression for M11L (vMyx-M11LKO-IL15Rα).

Embodiment 10 is the method of any one of embodiments 1-9, wherein the nuclear export inhibitor:
a) is a selective inhibitor of nuclear export (SINE);
b) binds to and/or inhibits exportin 1 (XPO1/CRM1);
c) binds to and/or inhibits a factor that binds to a nuclear export signal (NES);
d) binds to and/or inhibits a factor that binds to RAN, RAN-GTP, and/or RAN-GDP;
e) binds to and/or inhibits a factor that docks to the nuclear pore complex; and/or
f) binds to and/or inhibits a factor that mediates leucine-rich NES-dependent protein transport.

Embodiment 11 is the method of any one of embodiments 1-10, wherein the nuclear export inhibitor is not rapamycin or a structural analog thereof.

Embodiment 12 is the method of any one of embodiments 1-11, wherein the nuclear transport inhibitor is administered orally.

Embodiment 13 is the method of any one of embodiments 1-12, wherein the nuclear export inhibitor is at least one selected from the group consisting of selinexor, leptomycin A, leptomycin B, ratjadone A, ratjadone B, ratjadone C, ratjadone D, anguinomycin A, goniothalamin, piperlongumine, plumbagin, curcumin, valtrate, acetoxychavicol acetate, prenylcoumarin osthol, KOS 2464, PKF050-638, and CBS9106.

Embodiment 14 is the method of any one of embodiments 1-13, wherein the nuclear export inhibitor is selinexor and is administered at a dose per kilogram of subject body weight of between about 0.001 mg/kg and about 1000 mg/kg.

Embodiment 15 is the method of embodiment 13 or 14, wherein selinexor is administered in a tablet or a capsule.

Embodiment 16 is the method of any one of embodiments 13-15, wherein at least two doses of selinexor are administered.

Embodiment 17 is the method of any one of embodiments 1-16, wherein the MYXV is administered locally, systemically, intratumorally, intravenously, via injection, or via infusion.

Embodiment 18 is the method of any one of embodiments 1-17, wherein the MYXV is administered at a dose of from about 1×10³ focus-forming units (FFU) to about 1×10¹⁴ FFU.

Embodiment 19 is the method of any one of embodiments 1-18, wherein at least two doses of the MYXV are administered.

Embodiment 20 is the method of any one of embodiments 13-19, wherein the MYXV and selinexor are administered simultaneously or sequentially.

Embodiment 21 is the method of any one of embodiments 1-20, wherein the method increases replication of the MYXV in cancer cells of the subject by at least 10%.

Embodiment 22 is the method of any one of embodiments 1-21, wherein the method is effective to reduce average cancer load by at least 10%, and/or prolong average survival by at least 5% relative to an otherwise comparable treatment regimen that lacks either the MYXV or the nuclear export inhibitor as determined by a cohort study.

Embodiment 23 is the method of any one of embodiments 1-22, wherein the cancer load comprises a tumor volume or circulating hematological cancer cells.

Embodiment 24 is the method of any one of embodiments 1-23, wherein upon local administration of the MYXV, the MYXV reduces cancer growth at a site distal from the site of administration at least 10% more than in a corresponding method that lacks either the MYXV or the nuclear export inhibitor as determined by a cohort study.

Embodiment 25 is the method of any one of embodiments 2-24, wherein the heterologous transgene encodes a cytokine, interleukin, cell matrix protein, antibody, a checkpoint inhibitor, a multi-specific immune cell engager, or a functional fragment thereof.

Embodiment 26 is the method of any one of embodiments 2-25, wherein the heterologous transgene encodes an anti-PD-L1 antibody, decorin, IL-12, LIGHT, p14 FAST, TNF-α, a functional fragment thereof, or a combination thereof.

Embodiment 27 is the method of embodiment 26, wherein the multi-specific immune cell engager is a bispecific killer cell engager (BiKE) or a bispecific T cell engager (BiTE).

Embodiment 28 is the method of any one of embodiments 1-27, wherein the cancer is selected from the group consisting of a solid tumor, hematological tumor, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, colorectal adenocarcinoma, pancreatic cancer, and melanoma.

Embodiment 29 is the method of any one of embodiments 1-28, wherein the subject is immunocompetent, immunocompromised, or immunodeficient.

Embodiment 30 is the method of any one of embodiments 1-29, wherein the subject is a mammal.

Embodiment 31 is the method of any one of embodiments 1-30, wherein the subject is a human.

Embodiment 32 is the method of any one of embodiments 1-31, further comprising adsorbing the MYXV to a leukocyte ex vivo and administering the leukocyte to the subject.

Embodiment 33 is a therapeutic regimen comprising administering a myxoma virus (MYXV) and a nuclear export inhibitor to a subject with cancer, wherein the therapeutic regimen is effective to reduce average cancer load by at least 5% and/or prolong average survival by at least 5% relative to an otherwise comparable treatment regimen that lacks either the MYXV or the nuclear export inhibitor as determined by a cohort study.

Embodiment 34 is the therapeutic regimen of embodiment 33, wherein the nuclear export inhibitor is administered orally.

Embodiment 35 is the therapeutic regimen of embodiment 33 or 34, wherein the MYXV is a genetically modified MYXV.

Embodiment 36 is the therapeutic regimen of embodiment 35, wherein the genetically modified MYXV expresses a heterologous transgene, lacks expression for a gene, or a combination thereof.

Embodiment 37 is the therapeutic regimen of embodiments 35 or 36, wherein the genetically modified MYXV expresses IL15/IL15Rα, expresses human LIGHT, lacks expression for M11L, or a combination thereof.

Embodiment 38 is the therapeutic regimen of any one of embodiments 35-37, wherein the genetically modified MYXV expresses IL15/IL15Rα (v-Myx-IL15Rα).

Embodiment 39 is the therapeutic regimen of Embodiment 38, wherein the IL15/IL15Rα comprises an amino acid sequence with at least 90% homology to SEQ ID NO:1 or SEQ ID NO:9.

Embodiment 40 is the therapeutic regimen of any one of embodiments 35-39, wherein the genetically modified MYXV expresses human LIGHT (vMyx-hLIGHT).

Embodiment 41 is the therapeutic regimen of embodiment 40, wherein the human LIGHT comprises an amino acid sequence with at least 90% homology to SEQ ID NO:3.

Embodiment 42 is the therapeutic regimen of any one of embodiments 35-41, wherein the genetically modified MYXV lacks expression for M11L (vMyx-M11LKO).

Embodiment 43 is the therapeutic regimen of any one of embodiments 35-42, wherein the genetically modified MYXV expresses IL15/IL15Rα and lacks expression for M11L (vMyx-M11LKO-IL15Rα).

Embodiment 44 is the therapeutic regimen of any one of embodiments 33-43, wherein the nuclear export inhibitor:
a) is a selective inhibitor of nuclear export (SINE);
b) binds to and/or inhibits exportin 1 (XPO1/CRM1);
c) binds to and/or inhibits a factor that binds to a nuclear export signal (NES);
d) binds to and/or inhibits a factor that binds to RAN, RAN-GTP, and/or RAN-GDP;
e) binds to and/or inhibits a factor that docks to the nuclear pore complex; and/or
f) binds to and/or inhibits a factor that mediates leucine-rich NES-dependent protein transport.

Embodiment 45 is the therapeutic regimen of any one of embodiments 33-44, wherein the nuclear export inhibitor is not rapamycin or a structural analog thereof.

Embodiment 46 is the therapeutic regimen of any one of embodiments 33-45, wherein the nuclear export inhibitor is at least one selected from the group consisting of selinexor, leptomycin A, leptomycin B, ratjadone A, ratjadone B, ratjadone C, ratjadone D, anguinomycin A, goniothalamin, piperlongumine, plumbagin, curcumin, valtrate, acetoxychavicol acetate, prenylcoumarin osthol, KOS 2464, PKF050-638, and CBS9106.

Embodiment 47 is the therapeutic regimen of any one of embodiments 33-46, wherein the nuclear export inhibitor is selinexor and is administered at a dose per kilogram of subject body weight of between about 0.001 mg/kg and about 1000 mg/kg.

Embodiment 48 is the therapeutic regimen of embodiment 45 or 46, wherein selinexor is administered at a dose of between about 0.01 mg/kg and about 100 mg/kg.

Embodiment 49 is the therapeutic regimen of any one of embodiments 33-48, wherein the MYXV is administered locally, systemically, intratumorally, intravenously, via injection, or via infusion.

Embodiment 50 is the therapeutic regimen of any one of embodiments 33-49, wherein the MYXV is administered at a dose of from about 1×10³ focus-forming units (FFU) to about 1×10¹⁴ FFU.

Embodiment 51 is the therapeutic regimen of any one of embodiments 46-50, wherein the MYXV and selinexor are administered simultaneously or sequentially.

Embodiment 52 is the therapeutic regimen of any one of embodiments 33-51, wherein the cancer load comprises a tumor volume or concentration of circulating hematological cancer cells.

Embodiment 53 is the therapeutic regimen of any one of embodiments 33-52, wherein the therapeutic regimen is effective to reduce the average cancer load by at least 20% and/or prolong average survival by at least 20% relative to the otherwise comparable treatment regimen.

Embodiment 54 is the therapeutic regimen of any one of embodiments 33-53, wherein the MYXV is administered locally and the therapeutic regimen reduces incidence of metastasis at least 10% more than in a corresponding treatment regimen that lacks selinexor as determined by a cohort study and/or reduces cancer growth at a site distal from the site of administration at least 10% more than in a corresponding treatment regimen that lacks selinexor as determined by a cohort study.

Embodiment 55 is the therapeutic regimen of any one of embodiments 33-54, wherein the cancer is selected from the group consisting of a solid tumor, hematological tumor, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, colorectal adenocarcinoma, pancreatic cancer, and melanoma.

Embodiment 56 is the therapeutic regimen of any one of embodiments 33-55, wherein the subject is immunocompetent, immunocompromised, or immunodeficient.

Embodiment 57 is the therapeutic regimen of any one of embodiments 33-56, wherein the subject is a mammal.

Embodiment 58 is the therapeutic regimen of any one of embodiments 33-57, wherein the subject is a human.

Embodiment 59 is the therapeutic regimen of any one of embodiments 33-58, wherein the therapeutic regimen further comprises adsorbing the MYXV to a leukocyte ex vivo and administering the leukocyte to the subject.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Combination of an Oncolytic Virus and Nuclear Export Inhibitor

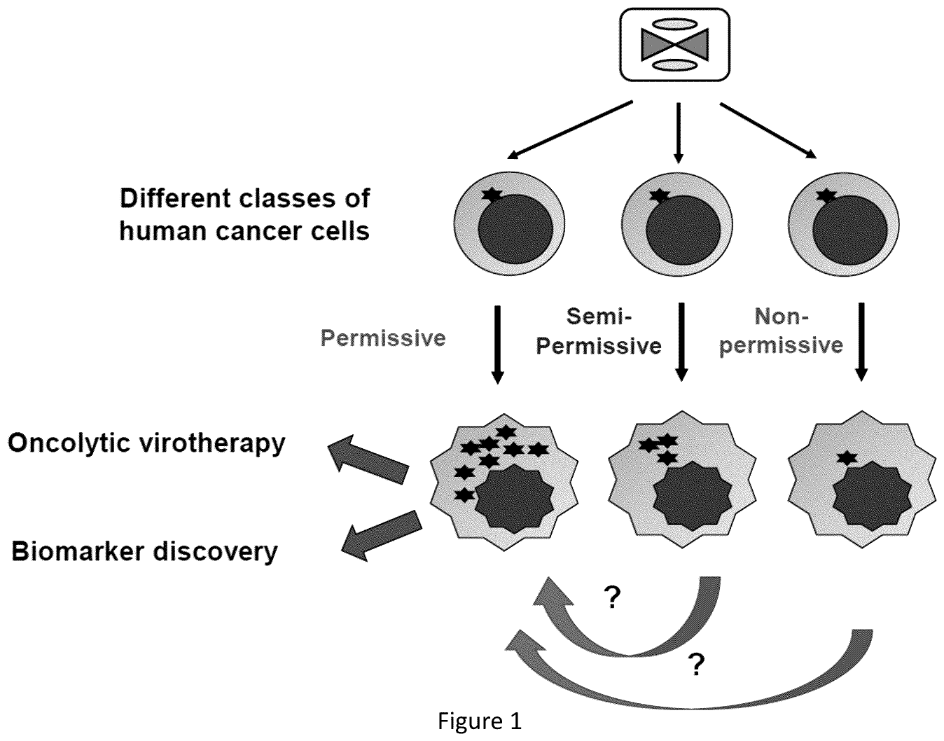

Oncolytic viruses (OVs) are cancer immunotherapy agents that selectively infect and kill cancer cells while sparing their normal counterparts. However, one of the limitations of OVs is their inability to successfully replicate, spread, and kill cancer cells in the complex tumor microenvironment (FIG. 1). The exportin-1 (CRM1/XPO1)-mediated nuclear export pathway can restrict oncolytic myxoma virus (MYXV) replication in diverse human cancer cell types.

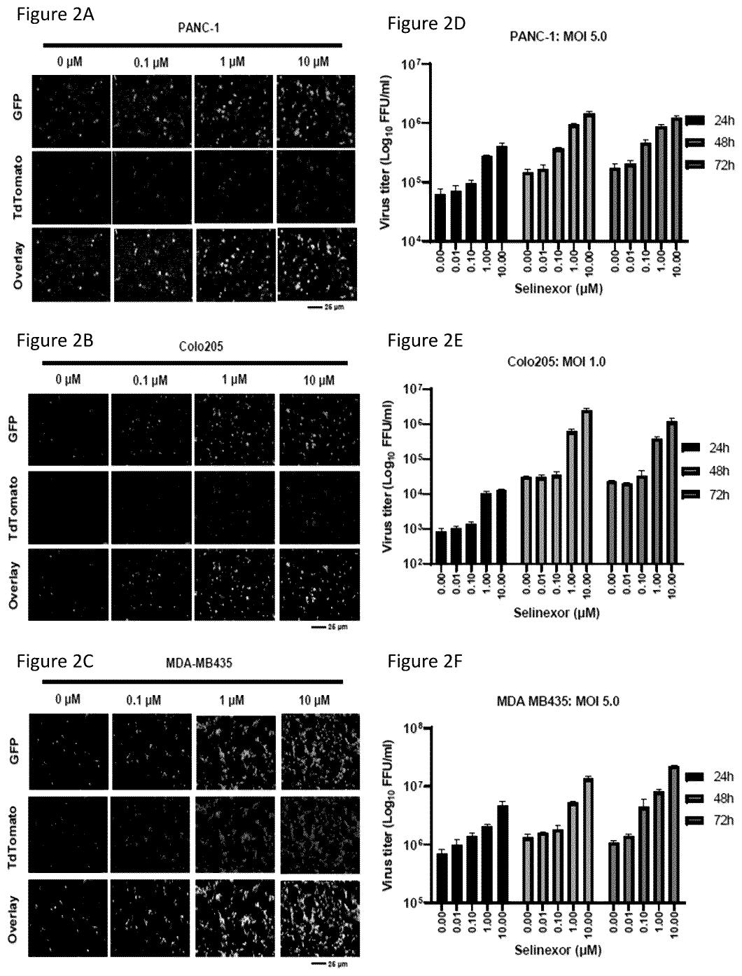

MYXV is a member of the Leporipoxvirus family of poxviruses. Inhibition of the CRM-1 pathway with an FDA-approved nuclear export inhibitor, selinexor, significantly enhanced MYXV replication in 2D and 3D cell cultures and in human xenograft tumor models in NSG mice. PANC-1 pancreatic cancer cells (FIG. 2A), Colo205 colon cancer cells (FIG. 2B), and MDA-MB435 melanoma cells (FIG. 2C) were treated with different concentrations of selinexor for 1 hour, infected with different MOI of vMyx-GFP-TdTomato for 1 hour, and replaced with fresh media containing the same doses of selinexor. Fluorescence images were taken 48 hours post-infection. PANC-1 (FIG. 2D), Colo205 (FIG. 2E), and MDA-MB435 (FIG. 2F) cells were harvested at different times post-infection to determine progeny virus production by titration assays on permissive RK13 cells. These results demonstrated that selinexor treatment significantly enhanced MYXV gene expression and replication in diverse human cancer cell lines.

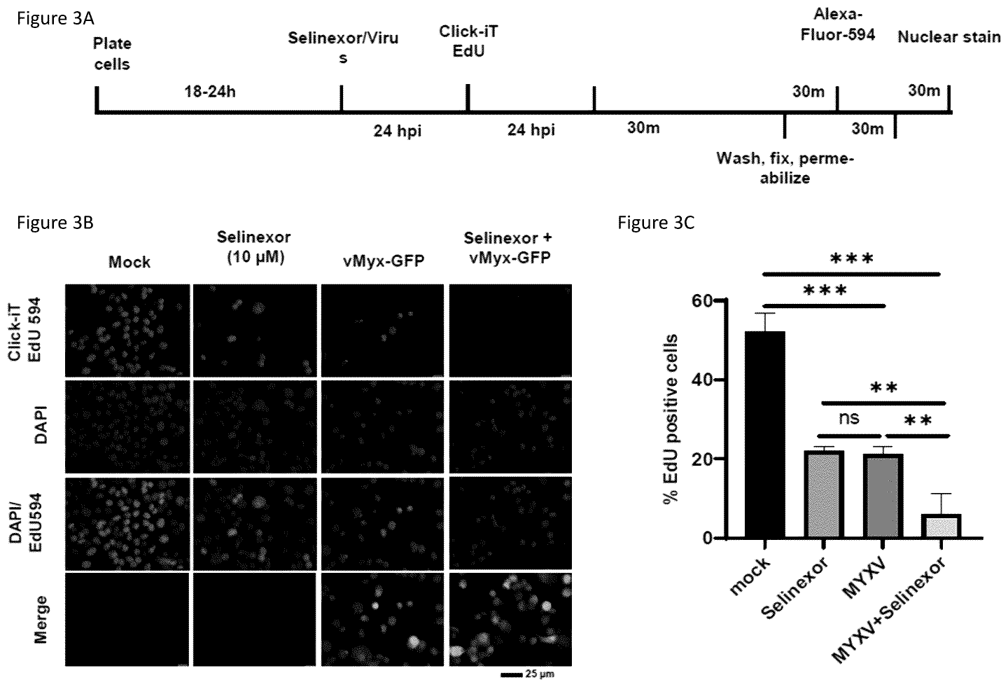

In another experiment, PANC-1 cells were left untreated (mock) or treated with selinexor, vMyx-GFP, or a combination of selinexor and vMyx-GFP, and a cell proliferation assay was performed using Click-iT EdU kit (FIG. 3A). Immunofluorescent images were taken (FIG. 3B) and the number of cells labeled with EdU 594 were quantified (FIG. 3C). These results demonstrated that combination of selinexor and oncolytic MYXV significantly reduced human cancer cell proliferation.

### Example 2: Modification of Myxoma Viruses

Based on the positive results with selinexor and MYXV, without being bound by a particular theory, it was examined whether virus-mediated activation of cell death pathways might further enhance the therapeutic potential of this combination therapy.

Selinexor was tested with modified MYXVs, including vMyx-hLIGHT, a recombinant MYXV which expresses human LIGHT (tumor necrosis factor superfamily member 14 (TNFSF14)), and vMyx-M11LKO, a MYXV construct lacking expression of anti-apoptotic protein M11L. These modifications to MYXV were chosen as they induce cancer cell death by activating different pathways.

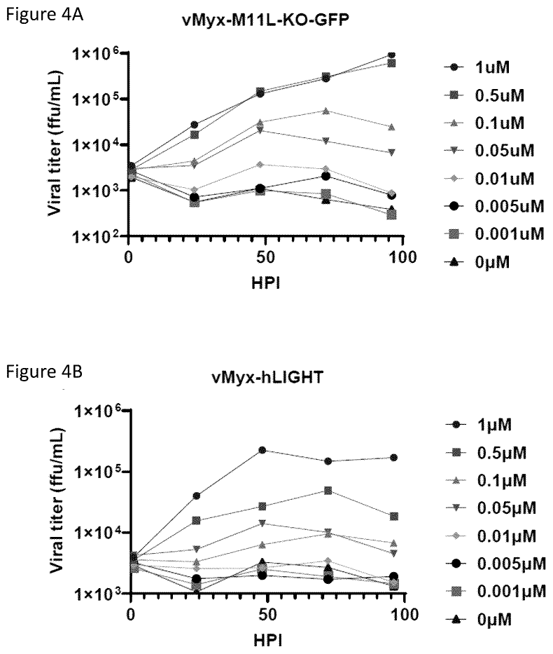

Selinexor treatment significantly enhanced the replication of both viruses in restricted cancer cell lines (FIG. 4). PANC-1 cells were treated with different concentration of selinexor for 1 hour, infected with an MOI of 1 with vMyx-M11L-KO or vMyx-hLIGHT viruses for 1 hour, and cultured in fresh media containing the same doses of selinexor without the MYXVs. Cells were harvested at different times post-infection to determine progeny virus production by titration assays on permissive RK13 cells (FIG. 4A and FIG. 4B).

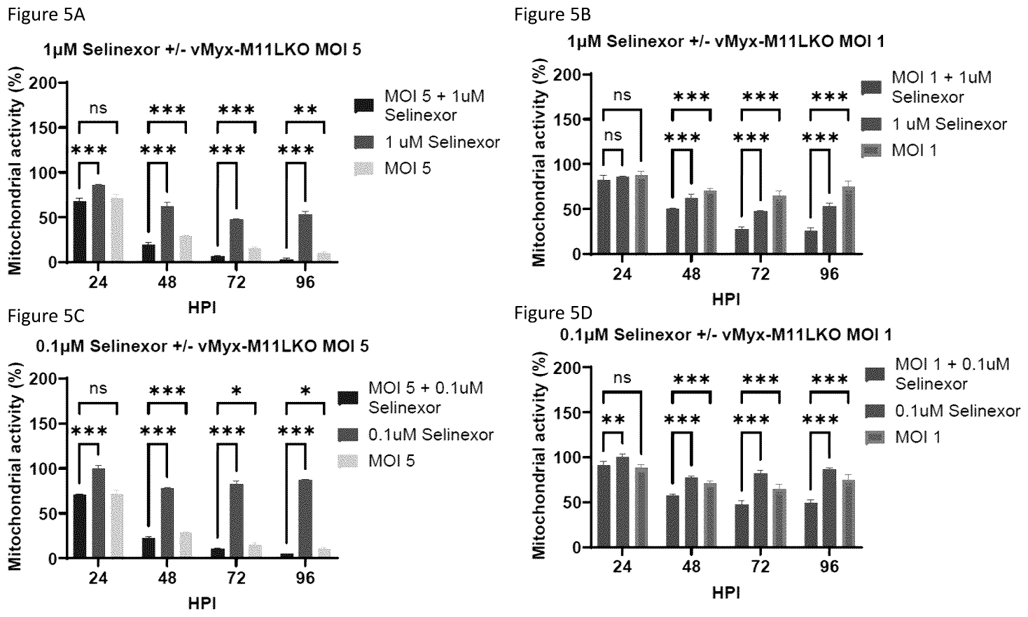

In the next set of experiments, the impact of selinexor and vMyx-M11L-KO co-administration on mitochondrial activity was examined. PANC-1 cells were seeded in 96 well plates and incubated overnight. The next day cells were left untreated (mock), treated with selinexor (1 μM or 0.1 μM), infected with vMyx-M11LKO (MOI of 1 or 5), or treated with a combination of selinexor and vMyx-M11LKO. Mitochondrial activity of the cells was examined over a span of four days (FIG. 5A-FIG. 5D). The results from these experiments demonstrated that combination of selinexor and oncolytic MYXV significantly reduced viability of human cancer cells.

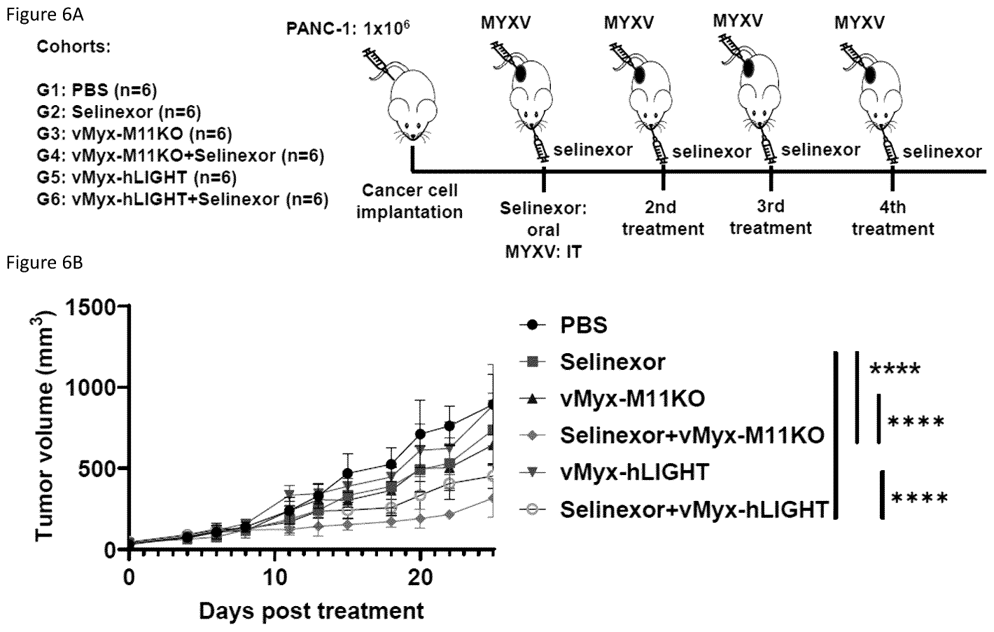

Anti-tumor activity was further investigated in a tumor xenograft model. NSG mice were inoculated with 1×10⁶ PANC-1 cells at day 0 via subcutaneous (SQ) injection in one of their flanks. After 2-3 weeks, animals received four doses of selinexor by oral gavage and four intratumoral (IT) injections of either vMyx-M11LKO or vMyx-hLIGHT (FIG. 6A). The combination of selinexor and oncolytic MYXVs significantly reduced PANC-1 xenograft tumor burden and prolonged survival in NSG mice for both vMyx-M11LKO and vMyx-hLIGHT (FIG. 6B).

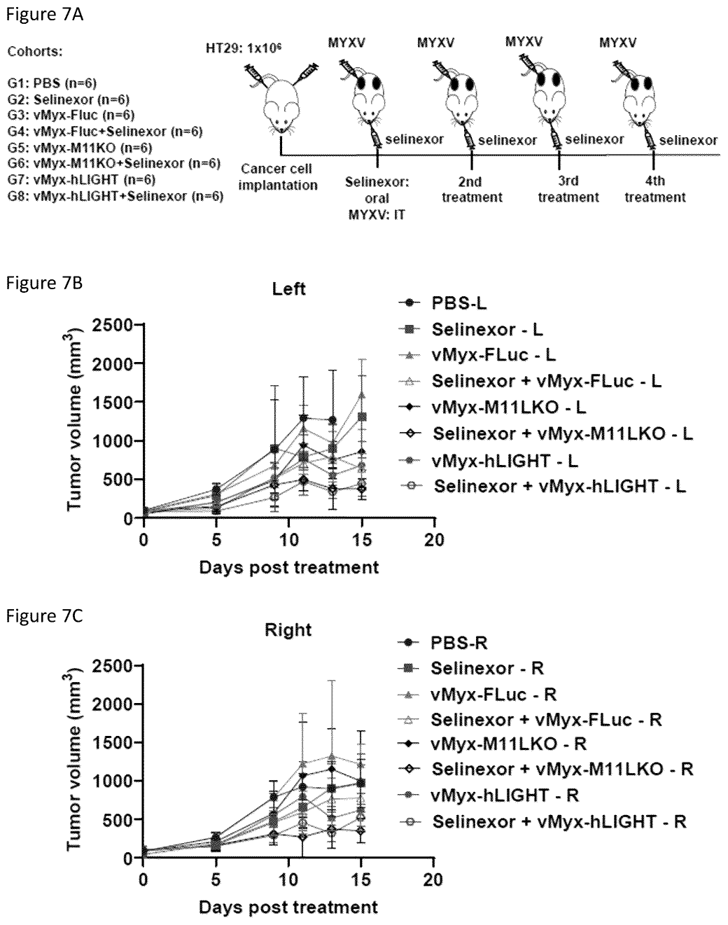

In a second set of experiments, NSG mice were inoculated with 1×10⁶ HT29 cells at day 0 via SQ injection on both flanks. After 2-3 weeks, the mice were separated into different treatment groups. Animals were administered PBS (control, G1), four doses of oral selinexor gavage (G2), four doses of IT injection of different viruses (vMyx-Fluc (expressing firefly luciferase), G3; vMyx-M11LKO, G5; vMyx-hLIGHT, G7) on the right flank, or a combination of oral selinexor and IT injection of viruses (vMyx-Fluc, G4; vMyx-M11LKO, G6; vMyx-hLIGHT, G8) on the right flank. The combination of selinexor with oncolytic MYXVs significantly reduced HT29 xenograft tumor volume in not only the right flank tumor that received the IT injection of MYXV (FIG. 7C) but also in the un-injected tumor in the left flank of the NSG mice (FIG. 7B).

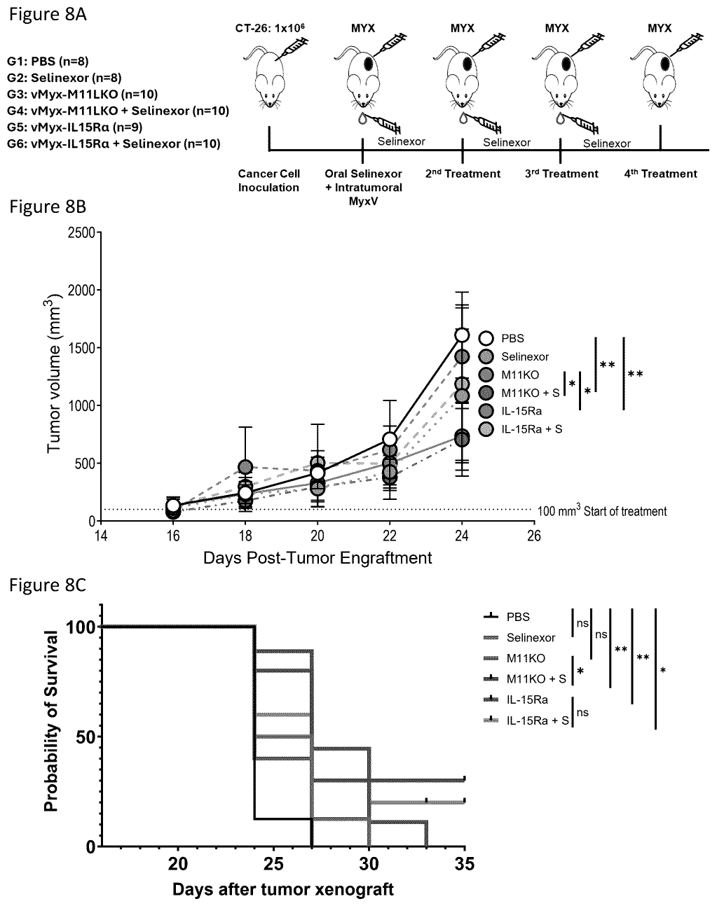

Encouraged by the results demonstrated by co-administration of selinexor and other MYXV, additional experiments were carried out using MYXV encoding interleukin-15 and interleukin-15 receptor alpha heterodimer complex (IL15/IL15Rα) (vMyx-IL15Rα). Immunocompetent BALB/c mice were inoculated with 1×10⁶ CT26 cells at day 0 via SQ injection on the right hind flank. After tumor volume reached ˜100 mm³ (day 16), animals received PBS (control, G1), oral selinexor (G2), IT vMyx-M11LKO or vMyx-IL15Rα (G3 and G5, respectively), or oral selinexor and IT vMyx-M11LKO or vMyx-IL15Rα (G4 and G6, respectively) (FIG. 8A). The combination of selinexor and oncolytic MYXVs significantly reduced CT26 xenograft tumor burden and prolong survival in immunocompetent BALB/c mice (FIG. 8B and FIG. 8C).

### Example 3: Effect of Doubly Modified Myxoma Virus

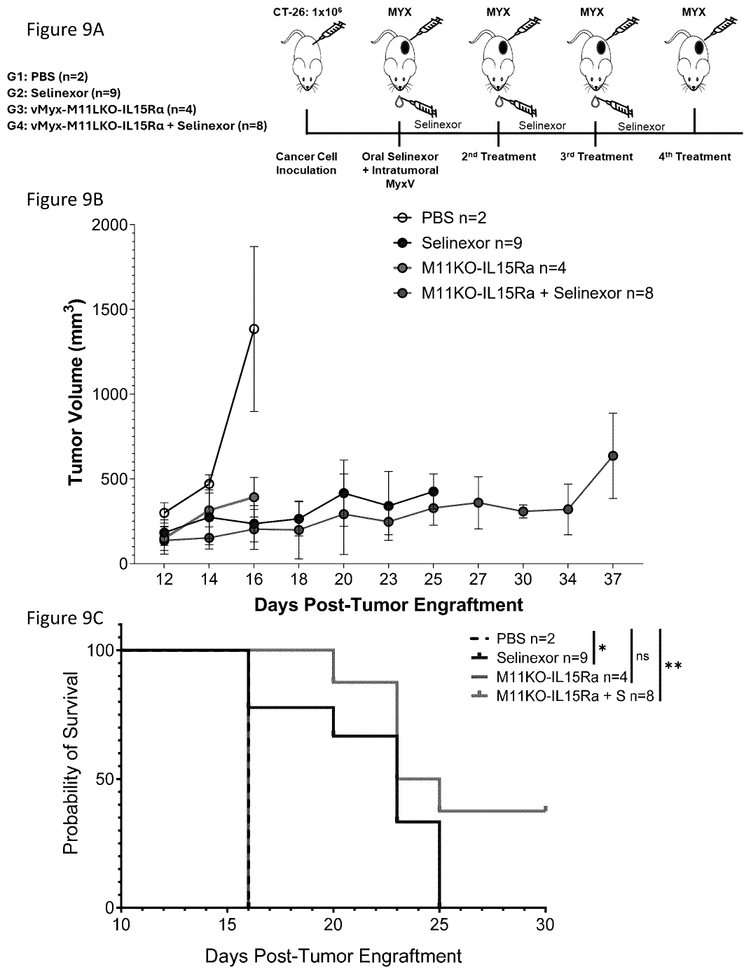

Given the beneficial effects resulting from the combination of selinexor and single-modified MYXV, the efficacy of MYXV that had been double modified was examined. As above, BALB/c mice were inoculated with 1×10⁶ CT26 cells at day 0 via SQ injection on the right hind flank. After tumor volume reached ˜100 mm³ (day 16), animals received PBS (G1, control), oral selinexor (G2), IT MYXV with M11L knocked out and expressing IL15Rα (v-Myx-M11LKO-IL15Rα, G3), or oral selinexor and IT v-Myx-M11LKO-IL15Rα (G4) (FIG. 9A).

The combination of selinexor and vMyx-M11LKO-IL15Rα not only significantly reduced CT26 xenograft tumor burden (FIG. 9B) but also greatly prolong survival in immunocompetent BALB/c mice (FIG. 9C).

These results demonstrate that oncolytic virus-mediated activation of cell death pathways can synergistically function with nuclear export inhibitors (e.g., selinexor) as a potential new anti-cancer combinatorial therapy.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating cancer in a subject in need thereof, the method comprising administering to the subject an effective amount of a genetically modified myxoma virus (MYXV) and an effective amount of a nuclear export inhibitor;
wherein the genetically modified MYXV expresses a heterologous transgene, does not express a native MYXV gene, or a combination thereof.

2. The method of claim 1, wherein the genetically modified MYXV expresses interleukin-15 and interleukin-15 receptor alpha heterodimer complex (IL15/IL15Rα), expresses human tumor necrosis factor superfamily member 14 (LIGHT), does not express M11L, or a combination thereof.

3. The method of claim 2, wherein the genetically modified MYXV expresses IL15/IL15Rα (v-Myx-IL15Rα).

4. The method of claim 3, wherein IL15/IL15Rα comprises an amino acid sequence with at least 90% homology to SEQ ID NO:1 or SEQ ID NO:9.

5. The method of claim 2, wherein the genetically modified MYXV expresses human LIGHT (v-Myx-hLIGHT).

6. The method of claim 5, wherein human LIGHT comprises an amino acid sequence with at least 90% homology to SEQ ID NO:3.

7. The method of claim 2, wherein the genetically modified MYXV does not express M11L (v-Myx-M11LKO).

8. The method of claim 2, wherein the genetically modified MYXV expresses IL15/IL15Rα and does not express M11L (vMyx-M11LKO-IL15Rα).

9. The method of claim 1, wherein the MYXV is administered locally, systemically, intratumorally, intravenously, via injection, or via infusion.

10. The method of claim 1, wherein the nuclear export inhibitor:
a) is a selective inhibitor of nuclear export (SINE);
b) binds to and/or inhibits exportin 1 (XPO1/CRM1);
c) binds to and/or inhibits a factor that binds to a nuclear export signal (NES);
d) binds to and/or inhibits a factor that binds to RAN, RAN-GTP, and/or RAN-GDP;
e) binds to and/or inhibits a factor that docks to the nuclear pore complex; and/or
f) binds to and/or inhibits a factor that mediates leucine-rich NES-dependent protein transport; and
wherein the nuclear export inhibitor is not rapamycin or a structural analog thereof.

11. The method of claim 1, wherein the nuclear export inhibitor is at least one selected from the group consisting of selinexor, leptomycin A, leptomycin B, ratjadone A, ratjadone B, ratjadone C, ratjadone D, anguinomycin A, goniothalamin, piperlongumine, plumbagin, curcumin, valtrate, acetoxychavicol acetate, prenylcoumarin osthol, KOS 2464, PKF050-638, and CBS9106.

12. The method of claim 11, wherein the nuclear export inhibitor is selinexor and is administered orally at a dose per kilogram of subject body weight of between about 0.001 mg/kg and about 1,000 mg/kg.

13. The method of claim 12, wherein selinexor is administered in a tablet or a capsule.

14. The method of claim 12, wherein at least two doses of selinexor are administered.

15. The method of claim 12, wherein the MYXV is administered at a dose of from about 1×10³ focus-forming units (FFU) to about 1×10¹⁴ FFU.

16. The method of claim 12, wherein at least two doses of the MYXV are administered.

17. The method of claim 12, wherein the MYXV and selinexor are administered simultaneously or sequentially.

18. The method of claim 12, wherein the method increases replication of the MYXV in cancer cells of the subject by at least 10% relative to replication of the MYXV in cancer cells of a subject not administered selinexor.

19. The method of claim 12, wherein the method is effective to reduce average cancer load by at least 10%, and/or prolong average survival by at least 5% relative to an otherwise comparable treatment regimen that lacks either the MYXV or the nuclear export inhibitor as determined by a cohort study,
wherein cancer load comprises a tumor volume or circulating hematological cancer cell count.

20. The method of claim 12, wherein upon local administration of the MYXV, the MYXV reduces cancer growth at a site distal from the site of administration at least 10% more than in a corresponding method that lacks either the MYXV or the nuclear export inhibitor as determined by a cohort study.

21. The method of claim 1, wherein the cancer is selected from the group consisting of a solid tumor, hematological tumor, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, retinoblastoma, colorectal adenocarcinoma, pancreatic cancer, and melanoma.

22. The method of claim 12, further comprising adsorbing the MYXV to a leukocyte ex vivo and administering the leukocyte to the subject.


SEQUENCE LISTING (14 sequences)

SEQ ID NO: 1 - protein, 252 aa, synthetic construct
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSMETDTLLLWVLLLWVPGSTGDGTTCPPPVSIEHADIRVKNYSVNSRERYVCNSGFKRKAGTSTLIECVINKNTNVAHWTTPSLKCIRDPS

SEQ ID NO: 2 - DNA, 759 bp, synthetic construct
  source 1..759: mol_type = other DNA
atgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtccatggagacagacaccctgctgctctgggtgctgctgctgtgggtgcccggctctaccggcgacggcaccacctgccctccccctgtgtccatcgagcacgccgacatcagagtgaagaactactccgtgaactctcgggagagatacgtgtgcaactccggcttcaagcggaaggccggcacctccaccctgatcgagtgcgtgatcaacaagaacaccaacgtggcccactggaccaccccttccctgaagtgcatccgggacccttcctga

SEQ ID NO: 3 - protein, 240 aa, synthetic construct
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 4 - DNA, 723 bp, synthetic construct
  source 1..723: mol_type = other DNA
atggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggccgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtgtga

SEQ ID NO: 5 - protein, 162 aa, synthetic construct
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 6 - DNA, 486 bp, synthetic construct
  source 1..486: mol_type = other DNA
atgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcc

SEQ ID NO: 7 - protein, 90 aa, synthetic construct
METDTLLLWVLLLWVPGSTGDGTTCPPPVSIEHADIRVKNYSVNSRERYVCNSGFKRKAGTSTLIECVINKNTNVAHWTTPSLKCIRDPS

SEQ ID NO: 8 - DNA, 273 bp, synthetic construct
  source 1..273: mol_type = other DNA
atggagacagacaccctgctgctctgggtgctgctgctgtgggtgcccggctctaccggcgacggcaccacctgccctccccctgtgtccatcgagcacgccgacatcagagtgaagaactactccgtgaactctcgggagagatacgtgtgcaactccggcttcaagcggaaggccggcacctccaccctgatcgagtgcgtgatcaacaagaacaccaacgtggcccactggaccaccccttccctgaagtgcatccgggacccttcctga

SEQ ID NO: 9 - protein, 252 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSMAPRRARGCRTLGLPALLLLLLLRPPATRGITCPPPMSVEHADIWVKSYSLYSRERYICNSGFKRKAGTSSLTECVLNKATNVAHWTTPS

SEQ ID NO: 10 - DNA, 766 bp, synthetic construct
  source 1..766: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctatggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgca

SEQ ID NO: 11 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 12 - DNA, 486 bp, synthetic construct
  source 1..486: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 13 - protein, 90 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPS

SEQ ID NO: 14 - DNA, 560 bp, synthetic construct
  source 1..560: mol_type = other DNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcaatggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgca